(19) United States
(12) Patent Application Publication
(10) Pub. No.: US 2016/0289319 A1
(43) Pub. Date: Oct. 6, 2016

(54) ANTI-CCL2 ANTIBODIES FOR TREATMENT OF SCLERODERMA

(71) Applicant: SHIRE HUMAN GENETIC THERAPIES, INC., Lexington, MA (US)

(72) Inventors: Paolo G.V. Martini, Boston, MA (US); Madhusudan Natarajan, Waban, MA (US); Patrick Anthony John Haslett, Somerville, MA (US); Albert Barnes Seymour, Westborough, MA (US)

(21) Appl. No.: 15/010,900

(22) Filed: Jan. 29, 2016

Related U.S. Application Data
(63) Continuation of application No. 14/403,025, filed on Nov. 21, 2014, now abandoned.
(60) Provisional application No. 61/650,149, filed on May 22, 2012.

(51) Int. Cl. C07K 16/24

(52) U.S. Cl. CPC C07K 16/24; C07K 2317/92; C07K 2317/21; A61K 2039/505

(57) ABSTRACT

The present invention provides, among other things, improved anti-CCL2 antibodies characterized with high affinity, potency, tissue selectivity and/or epitope specificity, and uses thereof, in particular, for treatment of scleroderma and related fibrotic and/or inflammatory diseases, disorders and conditions. In some embodiments, the present invention provides methods and compositions for treatment of scleroderma and related fibrotic and/or inflammatory diseases, disorders and conditions based on an anti-CCL2 antibody having an affinity of 10⁻¹²M or greater.

### CROSS REFERENCE TO RELATED APPLICATIONS

This application claims benefit under 35 USC §119(e) of U.S. Provisional Patent Application Ser. No. 61/650,149, filed May 22, 2012, which application is hereby incorporated by reference in its entirety.

### SEQUENCE LISTING

The present specification makes reference to a Sequence Listing submitted in electronic form as an ASCII.txt file named “2006685-0330_Sequences_ST25” on May 22, 2013. The .txt file was generated on May 14, 2013 and is 2 KB in size.

### BACKGROUND

Systemic sclerosis (scleroderma) is a clinically heterogeneous disorder of the connective tissue, resulting in hardening and tightening of the skin. It is an autoimmune—type of disease characterized by immune activation, vascular damage, and fibrosis. Major organ-based complications involving the lungs, heart, kidneys, and gastrointestinal tract can contribute to mortality and morbidity. The pathogenesis is unknown.

The feature most commonly associated with scleroderma is fibrosis—a buildup of collagen in the skin and organs. The buildup of collagen contributes to symptoms of the disorder, including hair loss, skin hardening and tightening, skin discoloration, joint pain, stiffness of fingers and joints, digestive tract problems and breathing complications (dry cough, shortness of breath, wheezing). Scleroderma may be classified into two major subgroups: limited cutaneous scleroderma and diffuse cutaneous scleroderma. In limited cutaneous scleroderma, fibrosis is mainly restricted to the hands, arms, and face. Diffuse cutaneous scleroderma is a rapidly progressing disorder that affects large areas of the skin and compromises one or more internal organs. Patients with limited cutaneous scleroderma have a relatively better long term prognosis than patients with diffuse cutaneous scleroderma. Widespread systemic scleroderma can damage the heart, kidney, lungs, or GI tract, which may cause death. Pulmonary fibrosis is the most common cause of death in patients with scleroderma.

1 Thus, scleroderma is an extremely debilitating disease with potentially fatal repercussions. There are about 50,000 patients in the US. The ratio of female patients to male patients is about 4:1. Current treatment methods are based only on symptomatic treatment and management of complications that arise through the course of the disease (e.g., corticosteroids, NSAIDs, and immune-suppressing medications such as Metotrexate and Cytoxan). There is no treatment shown to reverse or halt progression of disease. Therefore, there is a high unmet medical need for an effective treatment of scleroderma.

### SUMMARY OF THE INVENTION

The present invention provides improved methods and compositions for effective treatment of scleroderma, in particular, based on improved antibodies or binding proteins that can specifically bind to C—C chemokine ligand-2 (“CCL2”) with high affinity, potency, and/or epitope diversity to achieve robust biodistribution and/or tissue-specificity. CCL2 is known to be a validated target for scleroderma. Several studies have shown that scleroderma fibroblasts display increased constitutive expression of CCL2 mRNA and protein. In scleroderma skin sections, expression of CCL2 was detected in fibroblasts, keratinocytes, and mononuclear cells, whereas it was undetectable in normal skin (Galindo et al., Arthritis Rheum. 2001 June; 44(6):1382-6; Distler et al., Arthritis Rheum 2001 November; 44(11):2665-78; Lioyd et al., Exp Med. 1997 Apr. 7; 185(7): 1371-80; Yamamoto et al., J Dermatol Sci. 2001 June; 26(2): 133-9; Denton et al.; Trends Immunol. 2005 November; 26(11):596-602. Epub 2005 Sep. 15.). However, prior to the present invention, no effective treatment for scleroderma has been developed based on anti-CCL2 antibodies. The present inventors observe that high levels of CCL2 in plasma sequester anti-CCL2 antibodies injected intravenously, resulting in ineffective targeting of CCL2 in diseased tissues. To solve this problem, the present inventors contemplate the use of anti-CCL2 antibodies with high affinity administered in an amount sufficient to overcome the high levels of serum CCL2 leading to effective targeting of CCL2 in desired diseased tissues. In particular, the inventors contemplate “best in class” anti-CCL2 monoclonal antibody characterized with high binding affinity, tissue selectivity, epitope specificity and/or long half-life. Such inventive antibodies, once administered in vi, result in desired biodistribution and bioavailability such that they binds and blocks CCL2 signaling in target tissues reducing infiltration, inflammation and fibrosis, among other symptoms or features of scleroderma.

Thus, in one aspect, the present invention provides methods of treating scleroderma comprising administering to an individual who is suffering from or susceptible to scleroderma an effective amount of anti-CCL2 antibody, or fragment thereof, such that at least one symptom or feature of scleroderma in a target tissue is reduced in intensity, severity, or frequency, or has delayed onset.

In some embodiments, the at least one symptom or feature of scleroderma is selected from endothelial-cell damage, proliferation of basal-lamina layers, perivascular mononuclear-cell infiltration, fibrosis, derangement of visceral-organ architecture, rarefaction of blood vessels, hypoxia, and combination thereof.

In some embodiments, the target tissue is selected from the group consisting of skin, blood vessels, lung, heart, kidney, gastrointestinal tract (including liver), musculoskeletal system and combinations thereof. In some embodiments, the target tissue is lung. In some embodiments, the target tissue is heart.

In some embodiments, the individual is suffering from or susceptible to limited cutaneous scleroderma. In some embodiments, the individual is suffering from or susceptible to diffuse cutaneous scleroderma.

In some embodiments, the anti-CCL2 antibody, or fragment thereof, is administered parenterally. In some embodiments, the parenteral administration is selected from intravenous, intradermal, inhalation, transdermal (topical), subcutaneous, and/or transmucosal administration. In some embodiments, the parenteral administration is intravenous administration.

In some embodiments, the anti-CCL2 antibody, or fragment thereof, is administered orally.

In some embodiments, anti-CCL2 antibody, or fragment thereof, is administered bimonthly, monthly, triweekly, biweekly, weekly, daily, or at variable intervals.

In another aspect, the present invention provides use of an anti-CCL2 antibody, or fragment thereof, as described herein in the manufacture of a medicament for treatment of scleroderma, wherein the treatment comprises administering to an individual who is suffering from or susceptible to scleroderma an effective amount of the anti-CCL2 antibody, or fragment thereof, such that at least one symptom or feature of scleroderma in a target tissue is reduced in intensity, severity, or frequency, or has delayed onset.

In some embodiments, the present invention provides use of an anti-CCL2 antibody, or fragment thereof in the manufacture of a medicament for treating scleroderma as described herein, wherein the anti-CCL2 antibody, or fragment thereof, is characterized by binding affinity of stronger and/or greater than 10⁻¹² M (e.g., greater than 0.5×10⁻¹² M, 10⁻¹³ M, 0.5×10⁻¹³ M, 10⁻¹⁴ M, 0.5×10⁻¹⁴ M, or 10⁻¹⁵ M).

In some embodiments, an anti-CCL2 antibody, or fragment thereof, according to the invention is selected from the group consisting of intact IgG, F(ab′)₂, F(ab)₂, Fab′, Fab, scFvs, diabodies, triabodies and tetrabodies.

In some embodiments, the anti-CCL2 antibody, or fragment thereof, is a monoclonal antibody, optionally the anti-CCL2 antibody, or fragment thereof is a humanized monoclonal antibody, optionally the anti-CCL2 antibody, or fragment thereof is a human antibody.

In another aspect, the present invention provides methods of treating scleroderma comprising administering to an individual who is suffering from or susceptible to scleroderma an anti-CCL2 antibody, or fragment thereof, having a binding affinity of stronger and/or greater than 10⁻¹² M (e.g., greater than 0.5×10⁻¹² M, 10⁻¹³ M, 0.5×10⁻¹³ M, 10⁻¹⁴ M, 0.5×10⁻¹⁴ M, or 10⁻¹⁵ M).

In some embodiments, the anti-CCL2 antibody, or fragment thereof, is administered at a therapeutically effective dose and an administration interval such that the anti-CCL2 antibody, or fragment thereof, is distributed to one or more target tissues selected from the group consisting of skin, blood vessels, lung, heart, kidney, gastrointestinal tract (including liver), musculoskeletal system and combinations thereof. In some embodiments, the anti-CCL2 antibody, or fragment thereof, is administered at a therapeutically effective dose and an administration interval such that the anti-CCL2 antibody, or fragment thereof, is distributed to lung and/or heart.

In some embodiments, the administration interval is selected from bimonthly, monthly, triweekly, biweekly, weekly, daily, or at variable intervals.

In yet another aspect, the present invention provides methods of treating scleroderma comprising administering to an individual who is suffering from or susceptible to scleroderma an anti-CCL2 antibody, or fragment thereof, at a therapeutically effective dose and an administration interval such that the anti-CCL2 antibody, or fragment thereof, is distributed to lung and/or heart. In some embodiments, the anti-CCL2 antibody, or fragment thereof, is further distributed to skin, kidney, and/or liver.

In still another aspect, the present invention provides methods as disclosed in various embodiment above, wherein the anti-CCL2 antibody, or fragment thereof, is selected from the group consisting of intact IgG, F(ab′)₂, F(ab)₂, Fab′, Fab, scFvs, diabodies, triabodies and tetrabodies.

In some embodiments, the anti-CCL2 antibody, or fragment thereof, is a monoclonal antibody. In some embodiments, the anti-CCL2 antibody, or fragment thereof, is a humanized monoclonal antibody. In some embodiments, the anti-CCL2 antibody, or fragment thereof, is a human antibody.

Among other things, the present invention provides anti-CCL2 antibodies with high affinity. In some embodiments, the present invention provides an anti-CCL2 antibody, or fragment thereof, having a binding affinity of stronger and/or greater than 10⁻¹² M (e.g., greater than 0.5×10⁻¹² M, 10⁻¹³ M, 0.5×10⁻¹³ M, 10⁻¹⁴ M, 0.5×10⁻¹⁴ M, or 10⁻¹⁵ M).

In some embodiments, an anti-CCL2 antibody, or fragment thereof, according to the invention is selected from the group consisting of intact IgG, F(ab′)₂, F(ab)₂, Fab′, Fab, scFvs, diabodies, triabodies and tetrabodies.

In some embodiments, the anti-CCL2 antibody, or fragment thereof, is a monoclonal antibody.

In some embodiments, the anti-CCL2 antibody, or fragment thereof, is a humanized monoclonal antibody.

In some embodiments, the anti-CCL2 antibody, or fragment thereof, is a human antibody.

In another aspect, the present invention provides an anti-CCL2 antibody, or fragment thereof, as described herein for use in a method of treating scleroderma comprising a step of administering the anti-CCL2 antibody, or fragment thereof, to a subject, wherein the anti-CCL2 antibody, or fragment thereof, is characterized by a binding affinity of stronger and/or greater than 10⁻¹² M (e.g., greater than 0.5×10⁻¹² M, 10⁻¹³ M, 0.5×10⁻¹³ M, 10⁻¹⁴ M, 0.5×10⁻¹⁴ M, or 10⁻¹⁵ M).

In some embodiments, an anti-CCL2 antibody, or fragment thereof, according to the invention is selected from the group consisting of intact IgG, F(ab′)₂, F(ab)₂, Fab′, Fab, scFvs, diabodies, triabodies and tetrabodies.

In some embodiments, the anti-CCL2 antibody, or fragment thereof, is a monoclonal antibody, optionally the anti-CCL2 antibody, or fragment thereof is a humanized monoclonal antibody, optionally the anti-CCL2 antibody, or fragment thereof is a human antibody.

In yet another aspect, the present invention provides various compositions and kits containing an anti-CCL2 antibody described herein.

Other features, objects, and advantages of the present invention are apparent in the detailed description, drawings and claims that follow. It should be understood, however, that the detailed description, the drawings, and the claims, while indicating embodiments of the present invention, are given by way of illustration only, not limitation. Various changes and modifications within the scope of the invention will become apparent to those skilled in the art.

### BRIEF DESCRIPTION OF THE DRAWINGS

The drawings are for illustration purposes only not for limitation.

FIG. 1 illustrates an exemplary diagram depicting the Modified Rodnan Skin Score. Locations on the body where skin fibrosis is assessed are indicated.

FIG. 2 depicts an exemplary graph plotting serum and tissue concentration of CCL2 following equilibration.

FIG. 3 illustrates an exemplary diagram depicting CCL2 targeting in plasma and in diseased tissue.

### DEFINITIONS

In order for the present invention to be more readily understood, certain terms are first defined. Additional definitions for the following terms and other terms are set forth throughout the specification.

Affinity: As is known in the art. “affinity” is a measure of the tightness with which a particular ligand binds to (e.g., associates non-covalently with) and/or the rate or frequency with which it dissociates from, its partner. As is known in the art, any of a variety of technologies can be utilized to determine affinity. In many embodiments, affinity represents a measure of specific binding.

Affinity-matured (or affinity-matured antibody): As used herein, refers to an antibody with one or more alterations in one or more CDRs thereof which result an improvement in the affinity of the antibody for antigen, compared to a parent antibody which does not possess those alteration(s). In some embodiments, affinity matured antibodies will have nanomolar or even picomolar affinities for a target antigen. Affinity matured antibodies may be produced by any of a variety of procedures known in the art. Marks et al. BioTechnology 10:779-783 (1992) describes affinity maturation by V_{H} and V_{L} domain shuffling. Random mutagenesis of CDR and/or framework residues is described by: Barbas et al. Proc Nat. Acad. Sci, USA 91:3809-3813 (1994): Schier et al. Gene 169:147-155 (1995): Yelton et al. J. Immunol. 155:1994-2004 (1995): Jackson et al., J. Immunol. 154(7):3310-9 (1995); and Hawkins et al, J. Mol. Biol. 226:889-896 (1992).

Antibody: As used herein, the term “antibody” refers to a polypeptide consisting of one or more polypeptides substantially encoded by immunoglobulin genes or fragments of immunoglobulin genes. The recognized immunoglobulin genes include the kappa, lambda, alpha, gamma, delta, epsilon and mu constant region genes, as well as myriad immunoglobulin variable region genes. Light chains are typically classified as either kappa or lambda. Heavy chains are typically classified as gamma, mu, alpha, delta, or epsilon, which in turn define the immunoglobulin classes, IgG, IgM, IgA, IgD and IgE, respectively. A typical immunoglobulin (antibody) structural unit is known to comprise a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms “variable light chain” (V_{L}) and “variable heavy chain” (V_{H}) refer to these light and heavy chains respectively. An antibody can be specific for a particular antigen. The antibody or its antigen can be either an analyte or a binding partner. Antibodies exist as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}—CH₁ by a disulfide bond. The F(ab)′₂ may be reduced under mild conditions to break the disulfide linkage in the hinge region thereby converting the (Fab′)₂ dimer into an Fab′ monomer. The Fab′ monomer is essentially an Fab with part of the hinge region (see, Fundamental Immunology, W. E. Paul, ed., Raven Pres. N.Y. (1993), for a more detailed description of other antibody fragments). While various antibody fragments are defined in terms of the digestion of an intact antibody, one of ordinary skill in the art will appreciate that such Fab′ fragments may be synthesized de novo either chemically or by utilizing recombinant DNA methodology. Thus, the term “antibody,” as used herein also includes antibody fragments either produced by the modification of whole antibodies or synthesized de novo using recombinant DNA methodologies. In some embodiments, antibodies are single chain antibodies, such as single chain Fv (scFv) antibodies in which a variable heavy and a variable light chain are joined together (directly or through a peptide linker) to form a continuous polypeptide. A single chain Fv (“scFv”) polypeptide is a covalently linked V_{H}::V_{L} heterodimer which may be expressed from a nucleic acid including V_{H}- and V_{L}-encoding sequences either joined directly or joined by a peptide-encoding linker. (See, e.g., Huston, et al. (1988) Proc. Nat. Acad. Sci. USA, 85:5879-5883, the entire contents of which are herein incorporated by reference.) A number of structures exist for converting the naturally aggregated, but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Binding agent: As used herein, the term “binding agent” includes any naturally occurring, synthetic or genetically engineered agent, such as protein, that binds an antigen or a target protein or peptide. “Binding agent” is also referred to as “binding protein.” Binding agents can be derived from naturally occurring antibodies or synthetically engineered. A binding protein or agent can function similarly to an antibody by binding to a specific antigen to form a complex and elicit a biological response (e.g., agonize or antagonize a particular biological activity). Binding agents or proteins can include isolated fragments, “Fv” fragments consisting of the variable regions of the heavy and light chains of an antibody, recombinant single chain polypeptide molecules in which light and heavy chain variable regions are connected by a peptide linker (“scFv proteins”), and minimal recognition units consisting of the amino acid residues that mimic the hypervariable region. The term Binding Agent as used herein can also include antibody fragments either produced by the modification of whole antibodies or synthesized de novo using recombinant DNA methodologies. In some embodiments, antibodies are single chain antibodies, such as single chain Fv (scFv) antibodies in which a variable heavy and a variable light chain are joined together (directly or through a peptide linker) to form a continuous polypeptide. A single chain Fv (“scFv”) polypeptide is a covalently linked V_{H}::V_{L}, heterodimer which may be expressed from a nucleic acid including V_{H}- and V_{L}-encoding sequences either joined directly or joined by a peptide-encoding linker. (See, e.g., Huston, et al. (1988) Proc. Nat. Acad. Sci. USA. 85:5879-5883, the entire contents of which are herein incorporated by reference.) A number of structures exist for convening the naturally aggregated, but chemically separated light and heavy polypeptide chains from an antibody V region into an scFv molecule which will fold into a three dimensional structure substantially similar to the structure of an antigen-binding site. See, e.g. U.S. Pat. Nos. 5,091,513 and 5,132,405 and 4,956,778. In some embodiments, the term Binding Agent as used herein can also include antibody. See the definition of Antibody.

CDR: As used herein, refers to a complementarity determining region within an antibody variable region. There are three CDRs in each of the variable regions of the heavy chain and the light chain, which are designated CDR1, CDR2 and CDR3, for each of the variable regions. A “set of CDRs” or “CDR set” refers to a group of three or six CDRs that occur in either a single variable region capable of binding the antigen or the CDRs of cognate heavy and light chain variable regions capable of binding the antigen. Boundaries of CDRs have been defined differently depending on the system, of which several are known in the art (e.g., Kabat, Chothia, etc.).

Compound and Agent: The terms “compound” and “agent” are used herein interchangeably. They refer to any naturally occurring or non-naturally occurring (i.e., synthetic or recombinant) molecule, such as a biological macromolecule (e.g., nucleic acid, polypeptide or protein), organic or inorganic molecule, or an extract made from biological materials such as bacteria, plants, fungi, or animal (particularly mammalian, including human) cells or tissues. The compound may be a single molecule or a mixture or complex of at least two molecules.

Control: As used herein, the term “control” has its art-understood meaning of being a standard against which results are compared. Typically, controls are used to augment integrity in experiments by isolating variables in order to make a conclusion about such variables. In some embodiments, a control is a reaction or assay that is performed simultaneously with a test reaction or assay to provide a comparator. In one experiment, the “test” (i.e., the variable being tested) is applied. In the second experiment, the “control,” the variable being tested is not applied. In some embodiments, a control is a historical control (i.e., of a test or assay performed previously, or an amount or result that is previously known). In some embodiments, a control is or comprises a printed or otherwise saved record. A control may be a positive control or a negative control.

Dosing regimen: A “dosing regimen” (or “therapeutic regimen”), as that term is used herein, is a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length: in some embodiments, a dosing regimen comprises a plurality of doses and at least two different time periods separating individual doses.

Diagnosis: As used herein, the term “diagnosis” refers to a process aimed at determining if an individual is afflicted with a disease or ailment. In the context of the present invention, “diagnosis of scleroderma” refers to a process aimed at one or more of: determining if an individual is afflicted with scleroderma, identifying a scleroderma subtype (i.e., diffuse or limited cutaneous scleroderma), and determining the severity of the disease.

Effective amount: As used herein, the term “effective amount” refers to an amount of a compound or agent that is sufficient to fulfill its intended purpose(s). In the context of the present invention, the purpose(s) may be, for example: to modulate the cause or symptoms of scleroderma; and/or to delay or prevent the onset of scleroderma; and/or to slow down or stop the progression, aggravation, or deterioration of the symptoms of scleroderma; and/or to alleviate one or more symptoms associated with scleroderma; and/or to bring about amelioration of the symptoms of scleroderma, and/or to cure scleroderma.

Framework or framework region: As used herein, refers to the sequences of a variable region minus the CDRs. Because a CDR sequence can be determined by different systems, likewise a framework sequence is subject to correspondingly different interpretations. The six CDRs divide the framework regions on the heavy and light chains into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, FR1, for example, represents the first framework region closest to the amino terminal end of the variable region and 5′ with respect to CDR1, and FRs represents two or more of the sub-regions constituting a framework region.

Human antibody: As used herein, is intended to include antibodies having variable and constant regions generated (or assembled) from human immunoglobulin sequences. In some embodiments, antibodies (or antibody components) may be considered to be “human” even though their amino acid sequences include residues or elements not encoded by human germline immunoglobulin sequences (e.g., include sequence variations, for example that may (originally) have been introduced by random or site-specific mutagenesis in vitro or by somatic mutation in vivo), for example in one or more CDRs and in particular CDR3.

Humanized: As is known in the art, the term “humanized” is commonly used to refer to antibodies (or antibody components) whose amino acid sequence includes V_{H} and V_{L} region sequences from a reference antibody raised in a non-human species (e.g., a mouse), but also includes modifications in those sequences relative to the reference antibody intended to render them more “human-like”, i.e., more similar to human germline variable sequences. In some embodiments, a “humanized” antibody (or antibody component) is one that immunospecifically binds to an antigen of interest and that has a framework (FR) region having substantially the amino acid sequence as that of a human antibody, and a complementary determining region (CDR) having substantially the amino acid sequence as that of a non-human antibody. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor immunoglobulin) and all or substantially all of the framework regions are those of a human immunoglobulin consensus sequence. In some embodiments, a humanized antibody also comprises at least a portion of an immunoglobulin constant region (Fc), typically that of a human immunoglobulin constant region. In some embodiments, a humanized antibody contains both the light chain as well as at least the variable domain of a heavy chain. The antibody also may include a CH₁, hinge, CH₂, CH₃, and, optionally, a CH₄ region of a heavy chain constant region. In some embodiments, a humanized antibody only contains a humanized V_{L} region. In some embodiments, a humanized antibody only contains a humanized V_{H} region. In some certain embodiments, a humanized antibody contains humanized V_{H} and V_{L} regions.

Improve, increase, or reduce: As used herein, the terms “improve,” “increase” or “reduce,” or grammatical equivalents, indicate values that are relative to a baseline measurement, such as a measurement in the same individual prior to initiation of the treatment described herein, or a measurement in a control individual (or multiple control individuals) in the absence of the treatment described herein. A “control individual” is an individual afflicted with the same type and approximately the same severity of scleroderma as the individual being treated, who is about the same age as the individual being treated (to ensure that the stages of the disease in the treated individual and the control individual(s) are comparable).

Kit: As used herein, the term “kit” refers to any delivery system for delivering materials. Such delivery systems may include systems that allow for the storage, transport, or delivery of various diagnostic or therapeutic reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contains a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte Specific Reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components in a single container (e.g., in a single box housing each of the desired components). The term “kit” includes both fragmented and combined kits.

Normal: As used herein, the term “normal.” when used to modify the term “individual” or “subject” refers to an individual or group of individuals who does not have a particular disease or condition and is also not a carrier of the disease or condition. The term “normal” is also used herein to qualify a biological specimen or sample isolated from a normal or wild-type individual or subject, for example, a “normal biological sample.”

Nucleic Acid: As used herein the term “nucleic acid” refers to an oligonucleotide, nucleotide or polynucleotide, and fragments or portions thereof, and to DNA or RNA of genomic or synthetic origin that may be single or double stranded, and represents the sense or antisense strand.

Nucleic Acid Molecule: The terms “nucleic acid molecule” and “polynucleotide” are used herein interchangeably. They refer to a deoxyribonucleotide or ribonucleotide polymer in either single- or double-stranded form, and unless otherwise stated, encompass known analogs of natural nucleotides that can function in a similar manner as naturally occurring nucleotides. The terms encompasses nucleic acid-like structures with synthetic backbones, as well as amplification products.

Protein: In general, a “protein” is a polypeptide (i.e., a string of at least two amino acids linked to one another by peptide bonds). Proteins may include moieties other than amino acids (e.g., may be glycoproteins) and/or may be otherwise processed or modified. Those of ordinary skill in the art will appreciate that a “protein” can be a complete polypeptide chain as produced by a cell (with or without a signal sequence), or can be a functional portion thereof. Those of ordinary skill will further appreciate that a protein can sometimes include more than one polypeptide chain, for example linked by one or more disulfide bonds or associated by other means.

Sample: As used herein, the term “sample” encompasses any sample obtained from a biological source. The terms “biological sample” and “sample” are used interchangeably. A biological sample can, by way of non-limiting example, include skin tissue, liver tissue, kidney tissue, lung tissue, cerebrospinal fluid (CSF), blood, amniotic fluid, sera, urine, feces, epidermal sample, skin sample, cheek swab, sperm, amniotic fluid, cultured cells, bone marrow sample and/or chorionic villi. Cell cultures of any biological samples can also be used as biological samples. A biological sample can also be, e.g., a sample obtained from any organ or tissue (including a biopsy or autopsy specimen), can comprise cells (whether primary cells or cultured cells), medium conditioned by any cell, tissue or organ, tissue culture. In some embodiments, biological samples suitable for the invention are samples which have been processed to release or otherwise make available a nucleic acid for detection as described herein. Fixed or frozen tissues also may be used.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Suffering from: An individual who is “suffering from” a disease, disorder, and/or condition (e.g., scleroderma) has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, an individual who is susceptible to a disease, disorder, and/or condition (for example, scleroderma) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein associated with the disease, disorder, and/or condition; (4) habits and/or lifestyles associated with development of the disease, disorder, and/or condition; (5) a family history of the disease, disorder, and/or condition; (6) reaction to certain bacteria or viruses; (7) exposure to certain chemicals. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Treatment: As used herein, the term “treatment” (also “treat” or “treating”) refers to any administration of a therapeutic protein (e.g., administration of an anti-CCL2 monoclonal antibody or antigen binding fragment thereof) that partially or completely alleviates, ameliorates, relieves, inhibits, delays onset of, reduces severity of and/or reduces incidence of one or more symptoms or features of a particular disease, disorder, and/or condition (e.g., scleroderma, fibrosis or inflammation). Such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition.

### DETAILED DESCRIPTION OF THE INVENTION

The present invention provides, among other things, improved anti-CCL2 antibodies characterized with high affinity, potency, tissue selectivity and/or epitope specificity, and uses thereof, in particular, for treatment of scleroderma and related fibrotic and/or inflammatory diseases, disorders and conditions. In some embodiments, the present invention provides methods and compositions for treatment of scleroderma and related fibrotic and/or inflammatory diseases, disorders and conditions based on an anti-CCL2 antibody having an affinity of 10⁻¹² M or greater.

The present invention is, in part, based on the unique insights observed by the present inventors, that is, high affinity anti-CCL2 antibodies, particularly when administered in high doses, allow effective inhibition of CCL2 in affected tissues despite high levels of CCL2 in plasma. Embodiments of the invention include anti-CCL2 antibodies having an affinity of 10⁻¹² M or greater. Antibodies of such high affinity are particularly advantageous. Because of high circulating levels of CCL2 in plasma of patients with scleroderma, a large fraction of any anti-CCL2 antibody administered is likely to be sequestered by circulating CCL2. Without wishing to be bound by theory, a high affinity anti-CCL2 antibody can effectively neutralize CCL2 in affected tissue, in addition to neutralizing circulating CCL2, partly due to its ability to effectively compete off the receptor, CCR2, which has a binding affinity of 640 pM to CCL2. Thus, a high affinity anti-CCL2 antibody (e.g., an anti-CCL2 antibody with a binding affinity stranger than 60 pM) can effectively sequester CCL2 in diseased tissue preventing the binding between CCL2 and its receptor CCR2. As a result, less amount of high affinity anti-CCL2 antibody in diseased tissue may be required to achieve desired therapeutic effects.

Various aspects of the invention are described in detail in the following sections. The use of sections is not meant to limit the invention. Each section can apply to any aspect of the invention. In this application, the use of “or” means “and/or” unless stated otherwise.

### Scleroderma

Scleroderma, or systemic sclerosis, is generally considered a chronic systemic autoimmune disease characterized, among other things, fibrosis or hardening, vascular alterations, and autoantibodies. Without wishing to be bound by theory, it is thought that scleroderma is caused by a hyperactive autoimmune response trapped in a reinforcing amplification loop. For example, scleroderma is histologically characterized by inflammatory infiltrates of mononuclear cells, which in turn activate and are associated with increased collagen synthesis in the surrounding fibroblasts. In particular, activated macrophages produce TGF-beta and PDGF, which activate fibroblasts in the affected areas to produce high amounts of collagen.

T cells also appear to play a role in the disease process through activation of macrophages and the direct release of inflammatory pro-fibrogenic cytokines. In addition to collagen, the activated fibroblasts appear to secrete factors that recruit additional inflammatory cells to the affected areas, which release cytokines, which recruit further cytokine-releasing inflammatory cells, thereby leading to unregulated inflammation and tissue fibrosis.

Typically, monocytes/macrophages and T cells increase in both numbers and activation in the circulation and tissues of scleroderma patients. Tissue accumulation is both a cause and effect of microvascular injury, which is one of the early events in the pathogenesis of scleroderma. The microvascular injury is characterized by endothelial-cell damage, the proliferation of basal-lamina layers, occasional entrapment of peripheral-blood mononuclear cells in the vessel wall, and initial perivascular mononuclear-cell infiltrates. As the inflammatory cascade worsens, it is dominated by fibrosis, derangement of visceral organ architecture, rarefaction of blood vessels, and consequently, hypoxia. All of these factors and the continual recruitment of monocytes contributes to the maintenance of fibrosis

In some embodiments, scleroderma is also considered a connective tissue disease generally characterized with an excessive accumulation of Extracellular Matrix proteins in the skin and internal organs, vascular injury, and immunological abnormalities.

Many of the clinical manifestations of the disease are thought to involve a misregulation of vascular remodeling. One of the earliest symptoms of scleroderma is microvascular injury. This microvascular injury is thought to cause increased endothelial cell activation. Activated endothelial cells are believed to express adhesion molecules resulting in altered capillary permeability allowing migration of inflammatory cells through the endothelium and entrapment in the vessel wall. The immune activation is thought to contribute to sustained endothelial activation, which results in the breakdown of endothelial cells. This process is believed to contribute to the loss of elasticity and narrowing of the vessels commonly observed in scleroderma patients. Furthermore, it is thought that microvascular injury contributes to perivascular infiltrates of mononuclear cells in the dermis which is thought to contribute to the activation of fibroblasts and many of the associated hallmark symptoms of scleroderma. As fibrosis increases, permeability decreases. As a result, it becomes more difficult for antibodies to penetrate diseased tissues. Therefore, the affinity of anti-CCL2 antibodies becomes particularly important to keep antibodies localized.

Many of the clinical manifestations of the disease are generally thought to involve the misregulation of fibroblasts. The main function of fibroblasts is to maintain the structural integrity of connective tissues by continuously secreting precursors of the extracellular matrix. Fibroblasts provide a structural framework (stroma) for many tissues, play an important role in wound healing and are the most common cells of connective tissue in animals. Fibroblasts are morphologically heterogeneous with diverse appearances depending on their location and activity.

There are two major forms of scleroderma: limited systemic sclerosis/scleroderma and diffuse systemic sclerosis/scleroderma. In limited cutaneous scleroderma, the fibrosis of the skin is generally confined to the area proximal to the elbow. Patients with limited cutaneous scleroderma generally experience vascular impairment. Cutaneous and organ fibrosis generally progresses slowly in patients with limited scleroderma. Patients with diffuse scleroderma generally experience fibrosis of skin and organs that progresses more rapidly than in limited scleroderma and/or widespread inflammation and/or more severe internal organ involvement than is seen in limited scleroderma.

It is generally thought that interstitial lung disease, resulting in pulmonary fibrosis, is the leading cause of scleroderma related deaths (Ludwicka-Bradley. A., et al. Coagulation and autoimmunity in scleroderma interstitial lung disease. Semin Arthritis Rheum, 41(2), 212-22, 2011). Further complications resulting in scleroderma-related deaths include but are not limited to cancer, heart failure, pulmonary hypertension, kidney failure, and malabsorption, or any combination thereof.

Scleroderma is most commonly diagnosed by inspection of skin symptoms. Tests to diagnosis include but are not limited to visual and/or manual inspection of the skin, blood pressure testing, chest x-ray, lung CT, echocardiogram, urinalysis, skin biopsy, and blood tests including antinuclear antibody testing, anti-topoisomerase antibody testing, anti-centromere antibody testing, anti-U3 antibody testing, anti-RNA antibody testing, other types of antibody testing, erythrocyte sedimentation rate, and rheumatoid factor.

### Anti-CCL2 Antibodies

The present invention provides methods and compositions for treating scleroderma, and related fibrotic and/or inflammatory diseases, disorders and conditions, based on administration of anti-CCL2 antibodies, in particular, high affinity anti-CCL2 antibodies.

CCL2

CCL2 is a chemokine produced by a variety of cell types. It is also known as monocyte chemoattractant protein-1 (MCP-1). CCL2 is known to be a potent attractant for many cell types of the immune system, including but not limited to monocytes, CD4 and CD8 memory T lymphocytes and NK cells (Carulli, M. et al. Can CCL2 serum levels be used in risk stratification or to monitor treatment response in systemic sclerosis?Ann Rheum Dis, 67, 105-109, 2008. Yamamoto, 1′. Scleroderma—Pathophysiology. Eur J Dermatol, 19 (1), 14-24). CCL2 has been shown to promote leukocyte migration across endothelial monolayers, suggesting a role in the promotion of perivascular infiltrates of mononuclear cells (id.). CCL2 has also been shown to promote activation of tibroblasts and to upregulate Collagen type I mRNA expression in rat fibroblasts in vitro. Elevated CCL2 levels have been shown in patients with scleroderma and also in animal models of scleroderma (Id.). Specifically, increased CCL2 expression levels have been shown in scleroderma skin and increased CCL2 RNA and protein has been shown in scleroderma fibroblasts (Id.).

Human CCL2 is an 8.6 kDa protein containing 76 amino acid residues, the amino acid sequence of which is shown in Table 1. It is expressed by a variety of cell types, including monocytes, vascular endothelial cells, smooth muscle cells, certain epithelial cells, among others and binds its receptor CCR2. CCL2 belongs to the family of the CC chemokines which contains two cysteine residues that are adjacent (the adjacent cysteine residues underlined in Table 1).

TABLE 1

| | |
|---|---|
| Human CCL2 | MKVSAALLCLLLIAATFIPQGLAQPDAI |
| Protein Sequence | NAPVTCCYNFTNRKISVQRLASYRRITS |
| (GeneBank: | SKCPKEAVIFKTIVAKEICADPKQKWVQ |
| NP_002973) | DSMDHLDKQTQTPKT |
| | (SEQ ID NO: 1) |

CCL2 has also been purified, characterized, cloned and sequenced from non-human sources and can be recombinantly produced or chemically synthesized. As used herein, the term CCL2 encompasses any CCL2 proteins naturally-occurring in other species including, but not limited to, mouse, rats, primates, pigs, chickens, dogs, goats, sheeps, horses, camels, llama, to name but a few, and any recombinant or synthetic CCL2 that is substantially homologous or identical to human CCL2. In some embodiments, a CCL2 protein as used herein has a sequence at least 50%, 55%, 60%, 65%, 70%, 75%, 80%, 85%, 90%, 91%, 92%, 93%, 94%, 95%, 96%, 97%, 98%, 99% or more homologous to SEQ ID NO: 1. In some embodiments, a CCL2 protein as used herein has a sequence at least 50%, 55%, 60%, 65%, 70%, 75%, 80%, 85%, 90%, 91%, 92%, 93%, 94%, 95%, 96%, 96%, 97%, 98%, 99% or more identical to SEQ ID NO: 1. Typically, a CCL2 protein substantially homologous or identical to human CCL2 also retains substantial activity of human CCL2. “Percent (%) amino acid sequence identity” with respect to the CCL2 sequence identified herein is defined as the percentage of amino acid residues in a candidate sequence that are identical with the amino acid residues in the CCL2 sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, ALIGN or Megalign (DNASTAR) software. Those skilled in the art can determine appropriate parameters for measuring alignment, including any algorithms needed to achieve maximal alignment over the full length of the sequences being compared. Preferably, the WU-BLAST-2 software is used to determine amino acid sequence identity (Altschul et al., Methods in Enzynmology 266, 460-480 (1996); http://blast.wustl/edu/blast/README.html). WU-BLAST-2 uses several search parameters, most of which are set to the default values. The adjustable parameters are set with the following values: overlap span=1, overlap fraction=0.125, world threshold (T)=11. HSP score (S) and HSP S2 parameters are dynamic values and are established by the program itself, depending upon the composition of the particular sequence, however, the minimum values may be adjusted and are set as indicated above.

Any of the above described CCL2 proteins can be used to generate and identify mono-specific antibodies that specifically bind to CCL2. See the Anti-CCL2 Antibodies section below.

Anti-CCL2 Antibodies

CCL2 proteins described herein, or fragments thereof, can be used to generate antibodies by methods well known to those of skill in the art. As used herein, anti-CCL2 antibodies include any antibodies or fragments of antibodies that bind specifically to any epitopes of CCL2. As used herein, the term “antibodies” is intended to include immunoglobulins and fragments thereof which are specifically reactive to the designated protein or peptide, or fragments thereof. For example, the term “antibodies” includes intact monoclonal antibodies, polyclonal antibodies, single domain antibodies (e.g., shark single domain antibodies (e.g., IgNAR or fragments thereof)), and antibody fragments so long as they exhibit the desired biological activity. Suitable antibodies also include, but are not limited to, human antibodies, primatized antibodies, chimeric antibodies, bi-specific antibodies, humanized antibodies, conjugated antibodies (i.e., antibodies conjugated or fused to other proteins, radiolabels, cytotoxins), Small Modular ImmunoPharmaceuticals (“SMIPs™”), and antibody fragments.

As used herein, an “antibody fragment” includes a portion of an intact antibody, such as, for example, the antigen-binding or variable region of an antibody. Examples of antibody fragments include Fab, Fab′, F(ab′)2, and Fv fragments; triabodies; tetrabodies; linear antibodies; single-chain antibody molecules. The term “antibody fragment” also includes any synthetic or genetically engineered protein that acts like an antibody by binding to a specific antigen to form a complex. For example, antibody fragments include isolated fragments, “Fv” fragments, consisting of the variable regions of the heavy and light chains, recombinant single chain polypeptide molecules in which light and heavy chain variable regions are connected by a peptide linker (“scFv proteins”), and minimal recognition units consisting of the amino acid residues that mimic the hypervariable region.

Anti-CCL2 antibodies can be generated using methods well known in the art. For example, protocols for antibody production are described by Harlow and Lane, Antibodies: A Laboratory Manual, (1988) Typically, antibodies can be generated in mouse, rat, guinea pig, hamster, camel, llama, shark, or other appropriate host. Alternatively, antibodies may be made in chickens, producing IgY molecules (Schade et al., (1996) ALTEX 13(5):80-85). In some embodiments, antibodies suitable for the present invention are subhuman primate antibodies. For example, general techniques for raising therapeutically useful antibodies in baboons may be found, for example, in Goldenberg et al., international patent publication No. WO 91/11465 (1991), and in Losman et al., Int. J. Cancer 46: 310 (1990). In some embodiments, monoclonal antibodies may be prepared using hybridoma methods (Milstein and Cuello, (1983) Nature 305(5934):537-40.). In some embodiments, monoclonal antibodies may also be made by recombinant methods (U.S. Pat. No. 4,166,452, 1979).

Many of the difficulties associated with generating monoclonal antibodies by B-cell immortalization can be overcome by engineering and expressing antibody fragments in E. coli, using phage display. To ensure the recovery of high affinity, monoclonal antibodies a combinatorial immunoglobulin library must typically contain a large repertoire size. A typical strategy utilizes mRNA obtained from lymphocytes or spleen cells of immunized mice to synthesize cDNA using reverse transcriptase. The heavy- and light-chain genes are amplified separately by PCR and ligated into phage cloning vectors. Two different libraries are produced, one containing the heavy-chain genes and one containing the light-chain genes. Phage DNA is isolated from each library, and the heavy- and light-chain sequences are ligated together and packaged to form a combinatorial library. Each phage contains a random pair of heavy- and light-chain cDNAs and upon infection of E. coli directs the expression of the antibody chains in infected cells. To identify an antibody that recognizes the antigen of interest, the phage library is plated, and the antibody molecules present in the plaques are transferred to filters. The filters are incubated with radioactively labeled antigen and then washed to remove excess unbound ligand. A radioactive spot on the autoradiogram identifies a plaque that contains an antibody that binds the antigen. Cloning and expression vectors that are useful for producing a human immunoglobulin phage library can be obtained, for example, from STRATAGENE Cloning Systems (La Jolla, Calif.).

A similar strategy can be employed to obtain high-affinity scFv. See, e.g., Vaughn et al., Nat. Biotechnol., 14: 309 314 (1906). An scFv library with a large repertoire can be constructed by isolating V-genes from non-immunized human donors using PCR primers corresponding to all known V_{H}, V_{κ} and V_{λ} gene families. Following amplification, the V_{κ} and V_{λ}, pools are combined to form one pool. These fragments are ligated into a phagemid vector. The scFv linker, (Gly₄, Ser)₃, is then ligated into the phagemid upstream of the V_{L} fragment. The V_{H} and linker-V_{L} fragments are amplified and assembled on the J_{H} region. The resulting V_{H}-linker-V_{L} fragments are ligated into a phagemid vector. The phagemid library can be panned using filters, as described above, or using immunotubes (Nunc; Maxisorp). Similar results can be achieved by constructing a combinatorial immunoglobulin library from lymphocytes or spleen cells of immunized rabbits and by expressing the scFv constructs in P. pastoris. See, e.g., Ridder et al., Biotechnology. 13: 255 260 (1995). Additionally, following isolation of an appropriate scFv, antibody fragments with higher binding affinities and slower dissociation rates can be obtained through affinity maturation processes such as CDR3 mutagenesis and chain shuffling. See. e.g., Jackson et al., Br. J. Cancer, 78: 181 188 (1998): Osbourn et al., Immunotechnology, 2: 181 196 (1996).

Another form of an antibody fragment is a peptide coding for a single CDR. CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick et al., Methods: A Companion to Methods in Enzymology 2:106 (1991); Courtenay-Luck, “Genetic Manipulation of Monoclonal Antibodies.” in MONOCLONAL ANTIBODIES: PRODUCTION, ENGINEERING AND CLINICAL APPLICATION. Ritter et al. (eds.), pages 166 179 (Cambridge University Press 1995); and Ward et al., “Genetic Manipulation and Expression of Antibodies,” in MONOCLONAL ANTIBODIES: PRINCIPLES AND APPLICATIONS. Birch et al., (eds.), pages 137 185 (Wiley-Liss, Inc. 1995).

In some embodiments, antibodies suitable for the invention may include humanized or human antibodies. Humanized forms of non-human antibodies are chimeric Igs, Ig chains or fragments (such as Fv, Fab, Fab′, F(ab′)2 or other antigen-binding subsequences of Abs) that contain minimal sequence derived from non-human Ig. Generally, a humanized antibody has one or more amino acid residues introduced from a non-human source. These non-human amino acid residues are often referred to as “import” residues, which are typically taken from an “import” variable domain. Humanization is accomplished by substituting rodent complementarity determining regions (CDRs) or CDR sequences for the corresponding sequences of a human antibody (Riechmann et al., Nature 332(6162):323-7, 1988; Verhoeyen et al., Science. 239(4847):1534-6, 1988.). Such “humanized” antibodies are chimeric Abs (e.g. see U.S. Pat. Nos. 4,816,567; 5,693,762; and 5,225,539), wherein substantially less than an intact human variable domain has been substituted by the corresponding sequence from a non-human species. In some embodiments, humanized antibodies are typically human antibodies in which some CDR residues and possibly some FR residues are substituted by residues from analogous sites in rodent Abs. Humanized antibodies include human Igs (recipient antibody) in which residues from a CDR of the recipient are replaced by residues from a CDR of a non-human species (donor antibody) such as mouse, rat or rabbit, having the desired specificity, affinity and capacity. In some instances, corresponding non-human residues replace Fv framework residues of the human Ig. Humanized antibodies may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human Ig consensus sequence. The humanized antibody optimally also comprises at least a portion of an Ig constant region (Fc), typically that of a human Ig (Riechmann et al., Nature 332(6162):323-7, 1988; Verhoeyen et al., Science. 239(4847):1534-6, 1988.).

Human antibodies can also be produced using various techniques, including phage display libraries (Hoogenboom et al., Mol Immunol. (1991) 28(9):1027-37; Marks et al., J Mol Biol. (1991) 222(3):581-97) and the preparation of human monoclonal antibodies (Reisfeld and Sell, 1985, Cancer Surv. 4(1):271-90). Similarly, introducing human Ig genes into transgenic animals in which the endogenous Ig genes have been partially or completely inactivated can be exploited to synthesize human antibodies. Upon challenge, human antibody production is observed, which closely resembles that seen in humans in all respects, including gene rearrangement, assembly, and antibody repertoire (Fishwild et al., High-avidity human IgG kappa monoclonal antibodies from a novel strain of minilocus transgenic mice, Nat Biotechnol. 1996 July; 14(7):845-51; Lonberg et al., Antigen-specific human antibodies from mice comprising four distinct genetic modifications, Nature 1994 April 28:368(6474):856-9; Lonberg and Huszar, Human antibodies from transgenic mice, Int. Rev. Immunol. 1995; 13(1):65-93; Marks et al., By-passing immunization: building high affinity human antibodies by chain shuffling. Biotecimnology (NY) 1992 July; 10(7):779-83). In some embodiments, human anti-CCL2 antibodies are made by immunization of non-human animals engineered to make human antibodies in response to antigen challenge: e.g., immunization with human CCL2 (e.g., see U.S. Pat. Nos. 5,569,825; 6,150,584; and 6,596,541).

The use of high affinity anti-CCL2 antibodies to treat scleroderma is important. As described above, the binding affinity between CCL2 and the CCR2 receptor is high (i.e., 60 pM), and there is a high level of circulating CCL2 in plasma. Thus, majority of anti-CCL2 antibodies are likely to be sequestered in plasma once administered and only a small fraction may be localized to diseased target tissues. Therefore, anti-CCL2 antibodies are unlikely to be effective at competing CCL2 off of the receptor and inhibiting signaling in target tissue unless they also have a high binding affinity for CCL2. Furthermore, as scleroderma progresses, fibrosis increases and permeability of vasculature and access to target tissue decreases. The use of high affinity anti-CCL2 antibodies ensures that the antibodies retained at the target tissues are still capable of binding CCL2 and preventing interaction with its receptor.

Thus, in some embodiments, an anti-CCL2 antibody or fragment thereof suitable for the present invention has a binding affinity of or greater than approximately 500 nM, 100 nM, 10 nM, 1 nM, 500 pM, 100 pM, 50 pM, 10 pM, 1 pM, 500 fM, 400 fM, 300 fM, 200 fM, 100 fM, 50 fM, 10 fM, 1 fM. In some embodiments, an anti-CCL2 antibody or fragment thereof suitable for the present invention has a binding affinity ranging between approximately 500 nM and 1 fM, between 500 nM and 10 fM, between 500 nM and 100 fM, between 500 nM and 1 pM, between 10 nM and 1 fM, between 10 nM and 100 fM, between 10 nM and 1 pM, between 1 nM and 1 fM, between 1 nM and 100 fM, between 1 nM and 500 fM, between 1 nM and 1 pM, between 1 nM and 10 pM, between 1 nM and 50 pM, between 1 nM and 100 pM, between 1 nM and 500 pM.

### Biodistribution and Bioavailability

In various embodiments, once administered in vivo, an anti-CCL2 antibody according to the present invention may be delivered to various target tissues. Exemplary desired target tissues include, but are not limited, skin, blood vessels, lung, heart, kidney, gastrointestinal tract (including liver), esophagus, musculoskeletal system and combinations thereof.

In various embodiments, once administered in vivo, an anti-CCL2 antibody according to the present invention may achieve therapeutically or clinically effective levels or activities in various targets tissues described herein. As used herein, a therapeutically or clinically effective level or activity is a level or activity sufficient to confer a therapeutic effect in a target tissue. The therapeutic effect may be objective (i.e., measurable by some test or marker) or subjective (i.e., subject gives an indication of or feels an effect). For example, a therapeutically or clinically effective level or activity may be a protein level or activity that is sufficient to ameliorate symptoms associated with scleroderma or related diseases, disorders or conditions in the target tissue (e.g., CCL2 level). In some embodiments, an anti-CCL2 antibody described herein delivered according to the present invention may reduce CCL2 levels by at least 5%, 10%, 20%, 30%, 40%, 50%, 60%, 70%, 80%, 90%, 95% in the target tissue as compared to an untreated control or the pre-treatment state.

In some embodiments, an anti-CCL2 antibody described herein delivered according to the present invention may reduce the CCL2 serum level to less than about 1000 pg/ml, 900 pg/ml, 800 pg/ml, 700 pg/ml, 600 pg/ml, 500 pg/ml, 400 pg/ml, 300 pg/ml, 250 pg/ml, 200 pg/ml, 180 pg/ml, 160 pg/ml, 140 pg/ml, 120 pg/ml, 100 pg/ml, or less.

In general, once administered in vivo, an anti-CCL2 antibody according to the present invention have sufficiently long half time in serum and/or target tissues (e.g. skin, blood vessels, lung, heart, kidney, gastrointestinal tract (including liver), esophagus, or musculoskeletal system). In some embodiments, an anti-CCL2 antibody according to the present invention may have a half-life of at least approximately 30 minutes, 45 minutes, 60 minutes, 90 minutes, 2 hours, 3 hours, 4 hours, 5 hours, 6 hours, 7 hours, 8 hours, 9 hours, 10 hours, 12 hours, 16 hours, 18 hours, 20 hours, 25 hours, 30 hours, 35 hours, 40 hours, up to 3 days, up to 7 days, up to 14 days, up to 21 days or up to a month. In some embodiments, an anti-CCL2 antibody according to the present invention may retain detectable level or activity in serum and/or target tissues after 12 hours, 24 hours, 30 hours, 36 hours, 42 hours, 48 hours, 54 hours, 60 hours, 66 hours, 72 hours, 78 hours, 84 hours, 90 hours, 96 hours, 102 hours, or a week following administration. Detectable level or activity may be determined using various methods known in the art.

In certain embodiments, an anti-CCL2 antibody described herein achieves a concentration of at least 20 μg/ml, at least 15 μg/ml, at least 10 μg/ml, at least 7.5 μg/ml, at least 5 μg/ml, at least 2.5 μg/ml, at least 1.0 μg/ml or at least 0.5 μg/ml in the serum or targeted tissues following administration (e.g., intravenous) to such subject (e.g., one week, 3 days, 48 hours, 36 hours, 24 hours, 18 hours, 12 hours, 8 hours, 6 hours, 4 hours, 3 hours, 2 hours, 1 hour, 30 minutes, or less following administration (e.g., i.v.) to the subject).

### Treatment of Scleroderma and Related Diseases, Disorders or Conditions

Anti-CCL2 antibodies described herein may be used to effectively treat individuals suffering from or susceptible to scleroderma or related fibrotic, inflammatory diseases, disorders or conditions. The terms, “treat” or “treatment.” as used herein, refers to amelioration of one or more symptoms, prevention or delay of the onset of one or more symptoms, and/or lessening of the severity or frequency of one or more symptoms of the relevant disease, disorder or condition.

Various antibodies of the invention may be administered alone or in combination with other antibodies or therapeutic agents. In some embodiments, antibodies described herein may be administered alone or in conjunction with other therapeutic agents, such as those that are useful in treating fibrotic or inflammatory diseases, disorders or conditions. Such therapeutic agents include, but are not limited to, corticosteroids, NSAIDs, immune-suppressing drugs (e.g., Metotrexate and Cytoxan), small molecule immunomodulators, interferon receptor antibodies, anti-fibrotic drugs including D-penicillamine, colchicine, PUVA, relaxin, and cyclosporine and anti-TGFβ treatments, and endothelin receptor antagonists.

In some embodiments, antibodies described herein can be administered using conventional doses and delivery methods, such as those described for other, comparable therapeutic agents. Dosages to be administered can be determined by conventional procedures known to those of skill in the art. See, e.g., The Pharmacological Basis of Therapeutics, Goodman and Gilman, eds., Macmillan Publishing Co., New York. In general, effective dosages are those which are large enough to produce the desired effect, e.g., neutralizing CCL2 and/or blocking the binding of CCL2 to its cognate receptor. The dosage should not be so large as to cause adverse side effects, such as unwanted cross-reactions, anaphylactic reactions, and the like. Factors to be considered include the activity of the specific antibody/agent involved, its metabolic stability and length of action, mode and time of administration, drug combination, rate of excretion, and the age, body weight, general health, sex, diet, and severity of the particular disease-states of the host undergoing therapy.

Antibodies described herein can be administered in any dosing regimen that is therapeutically effective. In some embodiments, anti-CCL2 antibodies are administered at bimonthly, monthly, triweekly, biweekly, weekly, daily, or at variable intervals.

Antibodies described herein can be administered using any method of administration including parenteral and non-parenteral routes of administration. Parenteral routes include, e.g., intravenous, intraarterial, intraportal, intramuscular, subcutaneous, intraperitoneal, intraspinal, intrathecal, intracerebroventricular, intracranial, intrapleural or other routes of injection. Non-parenteral routes include, e.g., oral, nasal, transdermal, pulmonary, rectal, buccal, vaginal, ocular. Administration may also be by continuous infusion, local administration, sustained release from implants (gels, membranes or the like), and/or intravenous injection.

Scleroderma

In some embodiments, methods and compositions described herein can be used to treat a subject who is suffering or susceptible to all forms of scleroderma, including, the limited systemic sclerosis/scleroderma, the diffuse systemic sclerosis/scleroderma, and other forms of scleroderma. Limited systemic sclerosis/scleroderma typically involves cutaneous manifestations that mainly affect the hands, arms and face. It is also known as CREST syndrome in reference to the following complications: Calcinosis, Raynaud's phenomenon, Esophageal dysfunction, Sclerodactyly, and Telangiectasias. Additionally, pulmonary arterial hypertension may occur in up to one-third of patients, and is the most serious complication for this form of scleroderma. Diffuse systemic sclerosis/scleroderma is rapidly progressing and affects a large area of the skin and one or more internal organs, frequently the kidneys, esophagus, heart and lungs. Other forms of scleroderma include systemic sine scleroderma, which lacks skin changes, but has systemic manifestations, and two localized forms which affect the skin, but not the internal organs: morphea and linear scleroderma.

In some embodiments, treatment refers to partially or completely alleviation, amelioration, relief, inhibition, delaying onset, reducing severity and/or incidence of one or more symptoms associated with scleroderma, including but not limited to, endothelial-cell damage, proliferation of basal-lamina layers, perivascular mononuclear-cell infiltration, fibrosis, derangement of visceral-organ architecture, rarefaction of blood vessels, hypoxia, swelling of the fingers, dorsa, and forearms, sensations of coldness in the extremities, digital ulcers, elongation of nail folds, pitted bleeding of the nails, pitting scars on the nails, pulmonary hypertension, skin fibrosis, hair loss, skin tightness, skin hardness, hyperpigmentation, hypopigmentation, itching of the skin, carpal tunnel syndrome, muscle weakness, joint pain, joint stiffness, kidney fibrosis, esophageal fibrosis, mouth fibrosis, heart fibrosis, and lung fibrosis, liver fibrosis, muscle fibrosis, dry cough, shortness of breath, difficulty breathing, alveolitis, pneumonia, wheezing, bloating after meals, constipation, diarrhea, difficulty swallowing, gastric antral vascular ectasia, esophageal reflux, heartburn, fecal incontinence, flat white patches in the mouth, loss of attached gingival mucosa, gingival recession, diffuse widening of the periodontal ligament, dysphagia, inelasticity of the mouth, resorption of posterior ramus of the mandible, coronoid process, and condyle, cancer, heart failure, pulmonary hypertension, kidney failure, malabsorption, or any combination thereof, as compared to an untreated control or the pre-treatment state.

In some embodiments, treatment refers to partially or completely alleviation, amelioration, relief, inhibition, delaying onset, reducing severity and/or incidence of fibrosis. As used herein, the term “fibrosis” refers to the formation of an excess fibrous connective tissue in an organ or tissue. Without wishing to be bound by particular theory, it is thought that fibrosis may be caused by activation of certain fibroblast. Different subtypes of fibroblasts are known to perform diverse functions, even within a single tissue. For example, papillary fibroblasts of the upper layers of the skin produce thin collagen bundles and have a high rate of proliferation, whereas reticular fibroblasts from the deeper dermal layer of the skin produce thick collagen bundles and abundant versican, and promote rapid lattice contraction. Fibroblasts can be in a quiescent state or at varying stages of activation. During normal cellular function, fibroblasts become activated, for example, in response to injury to facilitate wound healing. Activated fibroblasts produce increased components of the extracellular matrix, including collagen and collagen modifying enzymes. In individuals with scleroderma, an increase in fibroblast activation is generally observed, accompanied by an overproduction of the ECM. This overproduction of the ECM is generally believed to cause fibrosis, the formation of excess fibrous connective tissue in an organ or tissue, which is a characteristic of scleroderma.

In some embodiments, treatment refers to partially or completely alleviation, amelioration, relief, inhibition, delaying onset, reducing severity and/or incidence of fibrosis in skin, kidney, gastrointestinal tract (including liver), blood vessels, gastrointestinal tract, musculoskeletal system, lung, and/or esophagus.

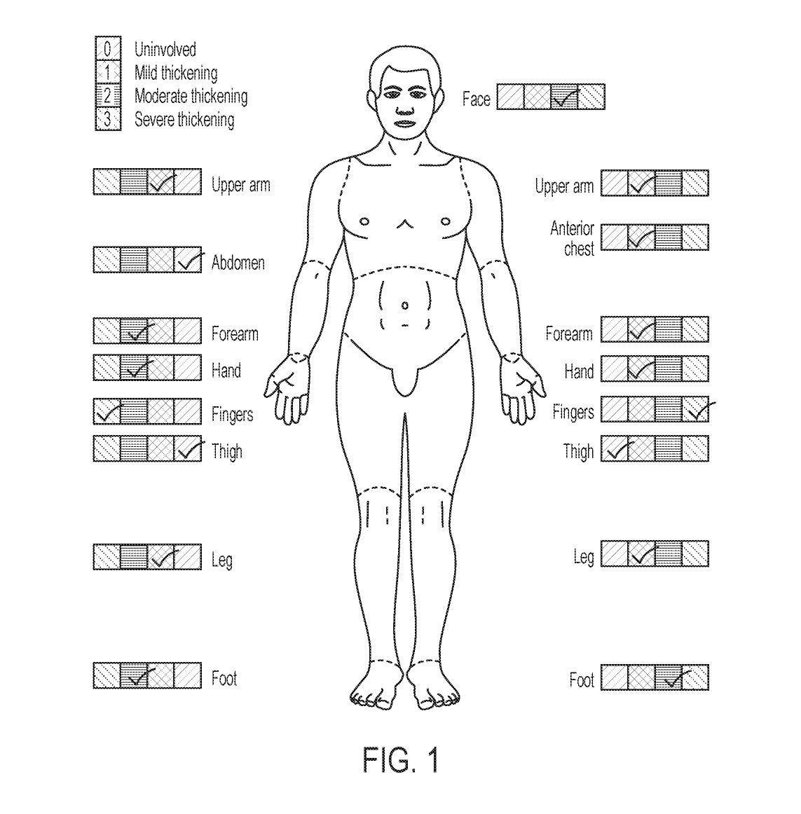

In some embodiments, treatment results in partially or completely alleviation, amelioration, relief, inhibition, delaying onset, reducing severity and/or incidence of skin fibrosis. Typically, skin fibrosis is associated with skin thickening, hardening, or formation of scan (e.g., keloid or burn scar, etc.). In some embodiments, skin fibrosis is assessed by Modified Rodnan Skin Score. For example, as illustrated in FIG. 1 uninvolved skin is given a score 0; mild thickening is given a score 1; moderate thickening is given a score 2; and severe thickening is given a score 3. In some embodiments, treatment results in a reduction of Modified Rodnan Skin Score by more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%, or more, as compared to the pre-treatment state. In some embodiments, treatment results in substantial elimination of skin fibrosis.

Without wishing to be bound by theory, it is also thought that activation of fibroblasts in scleroderma patients may be caused by the activation of the immune response by the production of cytokines. Examples of cytokines include but are not limited to TGF-β, CCL2, CTGF, ET-1. Fibroblast Growth Factor, IL-1, IL-4, IL-6, IL-12, IL-13, IL-17, MCP-1, MCP-3, and PDGF. Cytokines can be produced by pro-inflammatory cells of the immune system, for example activated T-cells, monocytes, or macrophages or, alternatively, cytokines can be produced by epithelial cells. One factor contributing to the activation of fibroblasts may be perivascular infiltrates of mononuclear cells in the dermis associated with increased capillary permeability. Alternative or additional means of fibroblast activation include interaction with the extracellular matrix and/or mechanical tension. Thus, in some embodiments, treatment of scleroderma patients according to the present invention results in reduction of the production of one or more pro-inflammatory cytokines, such as those described herein. In some embodiments, treatment results in a reduction of a pro-inflammatory cytokine (e.g., TGF-3, CCL2, CTGF, ET-1, Fibroblast Growth Factor, IL-1, IL-4, IL-6, IL-12, IL-13, IL-17, MCP-1, MCP-3, and/or PDGF) by more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%, or more, as compared to the pre-treatment state. Various methods for determining the level of cytokines are known in the art and can be used to practice the present invention.

In some embodiments, treatment results in reduced CCL2 serum levels. In some embodiments, treatment results in a reduction of CCL2 serum levels by more than 10%, more than 15%, more than 20%, more than 25%, more than 30%, more than 35%, more than 40%, more than 45%, more than 50%, more than 55%, more than 60%, more than 65%, more than 70%, more than 75%, more than 80%, more than 85%, more than 90%, more than 95%, or more, as compared to the pre-treatment state. In some embodiments, treatment results in a CCL2 serum level of less than about 800 pg/ml, 700 pg/ml, 600 pg/ml, 500 pg/ml, 400 pg/ml, 350 pg/ml, 300 pg/ml, 250 pg/ml, 200 pg/ml, 150 pg/ml, or 100 pg/ml. In some embodiments, treatment results in a CCL2 serum level comparable to that of a healthy control of substantially same age or developmental stage.

Fibrotic Diseases, Disorders or Condition

In addition to Sclerodera, methods and compositions according to the present invention can be used to treat fibrotic diseases, disorders or conditions in general including, but not limited to, multifocal fibrosclerosis, sclerodermatous graft-vs-host-disease, nephrogenic systemic fibrosis, organ specific fibrosis, and the like. Illustrative organ specific fibrotic disorders include, but are not limited to, pulmonary fibrosis, pulmonary hypertension, cystic fibrosis, asthma, chronic obstructive pulmonary disease, liver fibrosis, kidney fibrosis, NASH, and the like. Many fibrotic diseases, disorders or conditions have disordered and/or exaggerated deposition of extracellular matrix in affected tissues. Fibrosis may be associated with inflammation, occur as a symptom of underlying disease, and/or caused by surgical procedure or wound healing process. Unchecked fibrosis can result in destruction of the architecture of the underlying organ or tissue, commonly referred to as scarring.

NASH is usually a silent disease with few or no symptoms. Patients generally feel well in the early stages and only begin to have symptoms—such as fatigue, weight loss, and weakness once the disease is more advanced or cirrhosis develops. The progression of NASH can take years, even decades. The process can stop and, in some cases may even begin to reverse on its own without specific therapy. Or NASH can slowly worsen, causing scarring or fibrosis to appear and accumulate in the liver. As fibrosis worsens, cirrhosis develops in which the liver becomes seriously scarred, hardened, and unable to function normally. Not every person with NASH develops cirrhosis, but once serious scarring or cirrhosis is present, few treatments can halt the progression. A person with cirrhosis experiences fluid retention, muscle wasting, bleeding from the intestines, and liver failure. Liver transplantation is the only treatment for advanced cirrhosis with liver failure, and transplantation is increasingly performed in people with NASH. NASH ranks as one of the major causes of cirrhosis in America, behind hepatitis C and alcoholic liver disease.

Kidney (renal) fibrosis results from excessive formation of fibrous connective tissue in the kidney. Kidney fibrosis causes significant morbidity and mortality and leads to a need for dialysis or kidney transplantation. Fibrosis can occur in either the filtering or reabsorptive component of the nephron, the functional unit of the kidney. A number of factors may contribute to kidney scarring, particularly derangements of physiology involved in the autoregulation of glomerular filtration. This in turn leads to replacement of normal structures with accumulated extracellular matrix. A spectrum of changes in the physiology of individual cells leads to the production of numerous peptide and non-peptide fibrogens that stimulate alterations in the balance between extracellular matrix synthesis and degradation to favor scarring.

Inflammatory Diseases, Disorders or Conditions

In some embodiments, methods and compositions according to the present invention are used to treat inflammatory diseases, disorders or conditions including, but not limited to: Systemic Inflammatory Response (SIRS); Alzheimer's Disease (and associated conditions and symptoms including: chronic neuroinflammation, glial activation; increased microglia; neuritic plaque formation; and response to therapy); Amyotropic Lateral Sclerosis (ALS), arthritis (and associated conditions and symptoms including, but not limited to: acute joint inflammation, antigen-induced arthritis, arthritis associated with chronic lymphocytic thyroiditis, collagen-induced arthritis, juvenile arthritis; rheumatoid arthritis, osteoarthritis, prognosis and streptococcus-induced arthritis, spondyloarthopathies, gouty arthritis), asthma (and associated conditions and symptoms, including: bronchial asthma; chronic obstructive airway disease; chronic obstructive pulmonary disease, juvenile asthma and occupational asthma); cardiovascular diseases (and associated conditions and symptoms, including atherosclerosis; autoimmune myocarditis, chronic cardiac hypoxia, congestive heart failure, coronary artery disease, cardiomyopathy and cardiac cell dysfunction, including: aortic smooth muscle cell activation; cardiac cell apoptosis; and immunomodulation of cardiac cell function: diabetes and associated conditions and symptoms, including autoimmune diabetes, insulin-dependent (Type 1) diabetes, diabetic periodontitis, diabetic retinopathy, and diabetic nephropathy); gastrointestinal inflammations (and related conditions and symptoms, including celiac disease, associated osteopenia, chronic colitis, Crohn's disease, inflammatory bowel disease and ulcerative colitis); gastric ulcers; hepatic inflammations such as viral and other types of hepatitis, cholesterol gallstones and hepatic fibrosis, HIV infection (and associated conditions and symptoms, including degenerative responses, neurodegenerative responses, and HIV associated Hodgkin's Disease), Kawasaki's Syndrome (and associated diseases and conditions, including mucocutaneous lymph node syndrome, cervical lymphadenopathy, coronary artery lesions, edema, fever, increased leukocytes, mild anemia, skin peeling, rash, conjunctiva redness, thrombocytosis; multiple sclerosis, nephropathies (and associated diseases and conditions, including diabetic nephropathy, endstage renal disease, acute and chronic glomerulonephritis, acute and chronic interstitial nephritis, lupus nephritis, Goodpasture's syndrome, hemodialysis survival and renal ischemic reperfusion injury), neurodegenerative diseases (and associated diseases and conditions, including acute neurodegeneration, induction of IL-1 in aging and neurodegenerative disease, IL-1 induced plasticity of hypothalamic neurons and chronic stress hyperresponsiveness), ophtlialmopathies (and associated diseases and conditions, including diabetic retinopathy, Graves' opthalmopathy, and uveitis, osteoporosis (and associated diseases and conditions, including alveolar, femoral, radial, vertebral or wrist bone loss or fracture incidence, postmenopausal bone loss, mass, fracture incidence or rate of bone loss), otitis media (adult or pediatric), pancreatitis or pancreatic acinitis, periodontal disease (and associated diseases and conditions, including adult, early onset and diabetic); pulmonary diseases, including chronic lung disease, chronic sinusitis, hyaline membrane disease, hypoxia and pulmonary disease in SIDS; restenosis of coronary or other vascular grafts; rheumatism including rheumatoid arthritis, rheumatic Aschoff bodies, rheumatic diseases and rheumatic myocarditis; thyroiditis including chronic lymphocytic thyroiditis; urinary tract infections including chronic prostatitis, chronic pelvic pain syndrome and urolithiasis: immunological disorders, including autoimmune diseases, such as alopecia aerata, autoimmune myocarditis, Graves' disease, Graves opthalmopathy, lichen sclerosis, multiple sclerosis, psoriasis, systemic lupus erythematosus, systemic sclerosis, thyroid diseases (e.g. goiter and struma lymphomatosa (Hashimoto's thyroiditis, lymphadenoid goiter); sleep disorders and chronic fatigue syndrome and obesity (non-diabetic or associated with diabetes); resistance to infectious diseases, such as Leishmaniasis. Leprosy. Lyme Disease, Lyme Carditis, malaria, cerebral malaria, meningitis, tubulointerstitial nephritis associated with malaria), which are caused by bacteria, viruses (e.g. cytomegalovirus, encephalitis, Epstein-Barr Virus, Human Immunodeficiency Virus, Influenza Virus) or protozoans (e.g., Plasmodium falciparum, trypanosomes); response to trauma, including cerebral trauma (including strokes and ischemias, encephalitis, encephalopathies, epilepsy, perinatal brain injury, prolonged febrile seizures, SIDS and subarachnoid hemorrhage), low birth weight (e.g. cerebral palsy), lung injury (acute hemorrhagic lung injury. Goodpasture's syndrome, acute ischemic reperfusion), myocardial dysfunction, caused by occupational and environmental pollutants (e.g. susceptibility to toxic oil syndrome silicosis), radiation trauma, and efficiency of wound healing responses (e.g. burn or thermal wounds, chronic wounds, surgical wounds and spinal cord injuries); hormonal regulation including fertility/fecundity, likelihood of a pregnancy, incidence of preterm labor, prenatal and neonatal complications including preterm low birth weight, cerebral palsy, septicemia, hypothyroidism, oxygen dependence, cranial abnormality, early onset menopause; a subject's response to transplant (rejection or acceptance), acute phase response (e.g. febrile response), general inflammatory response, acute respiratory distress response, acute systemic inflammatory response, wound healing, adhesion, immunoinflammatory response, neuroendocrine response, fever development and resistance, acute-phase response, stress response, disease susceptibility, repetitive motion stress, tennis elbow, and pain management and response.

Biomarkers or Indicators for Patient Stratification, Treatment Monitoring and/or Optimization

In some embodiments, methods and compositions based on anti-CCL2 antibodies described herein can be used with biomarkers for patient stratification, treatment monitoring and/or optimization. In some embodiments, suitable biomarkers are differentially expressed biomarkers. As used herein, the term “differentially expressed biomarker” refers to a biomarker whose level of expression is different in a subject (or a population of subjects) afflicted with scleroderma relative to its level of expression in a healthy or normal subject (or a population of healthy or normal subjects). The term also encompasses a biomarker whose level of expression is different for a different disease subtype (i.e., limited cutaneous or diffuse cutaneous scleroderma). The term further encompasses a biomarker whose level of expression is different at different stages of the disease (e.g., mild or early scleroderma, severe or late scleroderma). Differential expression includes quantitative, as well as qualitative, differences in the temporal or cellular expression pattern of the biomarker. As described in greater details below, a differentially expressed biomarker, alone or in combination with other differentially expressed biomarkers, is useful in a variety of different applications in diagnostic, staging, therapeutic, drug development and related areas. The expression patterns of the differentially expressed biomarkers disclosed herein can be described as a fingerprint or a signature of scleroderma, scleroderma subtype, scleroderma stage and scleroderma disease severity and/or progression. They can be used as a point of reference to compare and characterize unknown samples and samples for which further information is sought. The term “decreased level of expression”, as used herein, refers to a decrease in expression of at least 10% or more, for example, 20%, 30%, 40%, or 50%, 60%, 70%, 80%, 90% or more, or a decrease in expression of greater than 1-fold, 2-fold, 3-fold, 4-fold, 5-fold, 10-fold, 50-fold, 100-fold or more as measured by one or more methods described herein. The term “increased level of expression”, as used herein, refers to an increase in expression of at least 10% or more, for example, 20%, 30%, 40%, or 50%, 60%, 70%, 80%, 90% or more or an increase in expression of greater than 1-fold, 2-fold, 3-fold, 4-fold, 5-fold, 10-fold, 50-fold, 100-fold or more as measured by one or more methods, such as method described herein.

Skin Gene Expression Analysis

Various methods for identifying differentially expressed biomarkers in scleroderma patients are known in the art and can be used to practice the present invention. For example, skin gene expression analysis can be a powerful tool for subsetting patients, identifying protein biomarkers and indicators of responsive patient subsets. In some embodiments, genes that are differentially regulated in patients with scleroderma can be identified by comparing transcriptional profiles of skin samples of healthy individuals with those having scleroderma. Further, gene transcripts that associate with severity of disease can be identified by including scleroderma patients at various stages of degree progression. Transcriptional profiles can be analyzed by microarray analysis, as has been described, for example, by Milano et al. in “Molecular Subsets in the Gene Expression Signatures of Scleroderma Skin” (PLOS One, 3:7, 1-18, 2008), the entirety of which is herein incorporated by reference. For example, microarray analysis can be performed on skin samples (e.g., forearm and back samples) from patients with diffuse scleroderma, limited scleroderma, morphea (a disease similar to scleroderma with no internal organ involvement) and healthy controls. To identify genes most highly associated with scleroderma, the genes that are most internally consistent between replicates and sample sites, while being the most variable between individuals, are selected for further analysis. Cluster analysis based on differential gene expression correlated with severity of scleroderma can be used to select genes affected by scleroderma.

It has been reported that differentially expressed exemplary genes in scleroderma can be clustered into 6 groups. The first group includes immunoglobulin genes expressed highly in a subset of patients with diffuse scleroderma and in patients with morphea, including but not limited to CCR2, CCL4, and IGLL1. The second group includes proliferation signature, including genes that are expressed only when the cell is dividing. Genes showing increased expression in this cluster include the cell-cycle regulated genes such as CKS1B, CDKS2, CDC2, MCM8 and E2F7. The existence of a proliferation signature is consistent with reports from skin biopsies demonstrating that cells of diffuse scleroderma tissue undergoing increased proliferation. The third group includes collagen and extracelluar matrix components, including but not limited to COL5A2, COL8A1, COL10A1, COL12A1. The fourth group includes genes typically associated with the presence of T-lymphocytes and macrophages, which are similarly expressed to the third group and include PTPRC, which is required for T-cell activation, as well as CD2 and CDW52, that are expressed on the surface of T lymphocytes. The fifth group includes genes showing low expression in diffuse scleroderma. These genes show higher expression levels in other biopsies and include WIF1, Tetranectin, IGFBP6, and IGFBP5, among others. The final group is a heterogeneous gene expression cluster that is high in limited scleroderma and a subset of diffuse scleroderma, including but not limited to, UTS2R, GALR3, PARD6G, PSEN1, PHOX2A, CENTG3, HCN4, KLF16, and GPR15G. Additional differentially expressed exemplary genes are described in Milano et al. in “Molecular Subsets in the Gene Expression Signatures of Scleroderma Skin” (PLOS One. 3:7, 1-18, 2008), the entirety of which is herein incorporated by reference.

Multi-Gene Signature as Surrogator Markers

Combinations of genes may be used as biomarkers. Exemplary methods for biomarker identification is provided in, for example, Farina et al., in “A Four-Gene Biomarker Predicts Skin Disease in Patients with Diffuse Cutaneous Systemic Sclerosis” (Arthritis Rheum. 62(2), 580-588, 2010), the entirety of which is incorporated herein by reference. Starting with targets such as TGFβ and interferon known to be regulated in scleroderma, Farina identified a four-gene biomarker, including the genes CTGF, THS1, COL4, and PAI1. The transcription of these four genes in combination was found to be highly correlated with Modified Rodnan Skin Score (mRSS) and highly predictive of diffuse scleroderma.

mRSS is used as one clinical marker of scleroderma. Typically, mRSS is assigned as shown in FIG. 1: uninvolved skin is assigned a score 0; mild thickening is given a score 1; moderate thickening is given a score 2; and severe thickening is given a score 3. Typically, a total mRSS score ranging from 0-51 can be determined based on a grading of 0-3 at 17 skin areas of a patient. mRSS can be used as indicators for diagnosis and monitoring treatment alone or in combination with other biomarkers.

Similar strategy can be used to identify and validate potential signature biomarkers for scleroderma. Specifically, gene transcripts identified as positively or negatively regulated in scleroderma are tested alone or in combination to identify biomarkers comprised of gene transcript(s) or combinations of gene transcripts that are most highly correlated with clinical markers of scleroderma. In addition to mRSS, other clinical markers can be used, such as the Health Assessment Questionnaire (HAQ-DI). Diffusing capacity of the lung for carbon monoxide (DLCO), or Forced Vital Capacity (FVC).

CCL2 Levels

CCL2 levels, for example, CCL2 serum levels, can be used as biomarker or indicators for determining disease severity, organ involvement, selecting appropriate treatment, monitoring disease progression and patient response. To determine CCL2 levels as biomarkers or indicators, CCL2 levels in the serum of patients at a variety of stages of scleroderma and unaffected individuals are determined. This can be done by assaying CCL2 protein levels in serum by, e.g., ELISA, and correlated with skin and other organ (e.g., lung, liver, kidney, oesophagus) involvement. Exemplary methods are described in Carulli et al. Ann Rheum Dis. 67:105-109, 2008.

CCL2 levels present in skin, such as from a biopsy, and/or serum can also be correlated with mRSS or other clinical markers, such as HAQ-DI, DLCO, or FVC.

Various biomarkers can be used alone or in combination, or alternatively, together with clinical diagnostic markers, such as mRSS, to stratify patients based on severity of scleroderma, selecting proper therapy or dosing regimen, evaluating the effectiveness of a therapy, monitoring responsiveness to therapy, prognosis for disease course, and measurement of disease progression in a subject. Typically, in such methods, levels of suitable biomarkers (e.g., such as those selected from various differentially expressed genes described herein and other known markers such as CCL2 levels) determined for a biological sample obtained from the subject from one or more time points are compared to the levels from the subject from one or more other time points. For example, biomarker levels may be measured before or at the beginning of a treatment course. Biomarker levels may be measured at one or more time points throughout the course of treatment and compared with the level before the treatment or from an earlier time point of a treatment course. Identification or selection of appropriate treatment, determining if a patient has positive response to a treatment and/or optimization of the treatment can be determined based on the evaluation of biomarkers.

### Pharmaceutical Compositions

The present invention also provides compositions comprising one or more provided antibodies. In some embodiments the present invention provides at least one antibody and at least one pharmaceutically acceptable excipient. Such pharmaceutical compositions may optionally comprise and/or be administered in combination with one or more additional therapeutically active substances. In some embodiments, provided pharmaceutical compositions are useful in medicine. In some embodiments, provided pharmaceutical compositions are useful as prophylactic agents (i.e., vaccines) in the treatment or prevention of scleroderma or of negative ramifications associated or correlated with scleroderma. In some embodiments, provided pharmaceutical compositions are useful in therapeutic applications, for example in individuals suffering from or susceptible to scleroderma. In some embodiments, pharmaceutical compositions are formulated for administration to humans.

For example, pharmaceutical compositions provided here may be provided in a sterile injectable form (e.g., a form that is suitable for subcutaneous injection or intravenous infusion). For example, in some embodiments, pharmaceutical compositions are provided in a liquid dosage form that is suitable for injection. In some embodiments, pharmaceutical compositions are provided as powders (e.g., lyophilized and/or sterilized), optionally under vacuum, which are reconstituted with an aqueous diluent (e.g., water, buffer, salt solution, etc.) prior to injection. In some embodiments, pharmaceutical compositions are diluted and/or reconstituted in water, sodium chloride solution, sodium acetate solution, benzyl alcohol solution, phosphate buffered saline, etc. In some embodiments, powder should be mixed gently with the aqueous diluent (e.g., not shaken).

In some embodiments, provided pharmaceutical compositions comprise one or more pharmaceutically acceptable excipients (e.g., preservative, inert diluent, dispersing agent, surface active agent and/or emulsifier, buffering agent, etc.). In some embodiments, pharmaceutical compositions comprise one or more preservatives. In some embodiments, pharmaceutical compositions comprise no preservative.

In some embodiments, pharmaceutical compositions are provided in a form that can be refrigerated and/or frozen. In some embodiments, pharmaceutical compositions are provided in a form that cannot be refrigerated and/or frozen. In some embodiments, reconstituted solutions and/or liquid dosage forms may be stored for a certain period of time after reconstitution (e.g., 2 hours, 12 hours, 24 hours, 2 days, 5 days, 7 days, 10 days, 2 weeks, a month, two months, or longer). In some embodiments, storage of antibody compositions for longer than the specified time results in antibody degradation.

Liquid dosage forms and/or reconstituted solutions may comprise particulate matter and/or discoloration prior to administration. In some embodiment, a solution should not be used if discolored or cloudy and/or if particulate matter remains after filtration.

Compositions of the pharmaceutical compositions described herein may be prepared by any method known or hereafter developed in the art of pharmacology. In some embodiments, such preparatory methods include the step of bringing active ingredient into association with one or more excipients and/or one or more other accessory ingredients, and then, if necessary and/or desirable, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to a dose which would be administered to a subject and/or a convenient fraction of such a dose such as, for example, one-half or one-third of such a dose.

Relative amounts of active ingredient, pharmaceutically acceptable excipient, and/or any additional ingredients in a pharmaceutical composition in accordance with the invention may vary, depending upon the identity, size, and/or condition of the subject treated and/or depending upon the route by which the composition is to be administered. By way of example, the composition may comprise between 0.1% and 100% (w/w) active ingredient.

Pharmaceutical compositions of the present invention may additionally comprise a pharmaceutically acceptable excipient, which, as used herein, may be or comprise solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21st Edition, A. R. Gennaro, (Lippincott, Williams & Wilkins, Baltimore. Md., 2006) discloses various excipients used in formulating pharmaceutical compositions and known techniques for the preparation thereof. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

### EXAMPLES

### Example 1

### Preparation of High Affinity Anti-CCL2 Antibodies

This example illustrates preparation of high affinity anti-CCL2 antibodies. As described above, various methods are available to generate and select antibodies with desired specificities and binding affinities.

In this particular example, the anti-CCL2 antibody is composed of a complete human antibody comprising two full-length antigen binding arms. Transgenic mice expressing human antibody genes are initially immunized with purified human recombinant CCL2 in complete Freund's adjuvant via subcutaneous injection. Following the initial immunization, each of the mice receives an additional subcutaneous injection once a week for three weeks. Splenocytes are harvested from mice with high antibody titres, as determined by ELISA, and fused to a mouse myeloma cell line as follows. Single cell suspensions of splenocytes from immunized mice are fused to one-fourth the number of non-secreting mouse myeloma cells with 50% PEG. Cells are plated at approximately 1×10⁵/well in flat bottom microtiter plates, followed by a one week incubation. Individual wells are then screened by ELISA for human anti-CCL2 monoclonal IgG antibodies. Once extensive hybridoma growth occurs, the antibody-secreting hybridomas are replated, screened again and, if still positive for human IgG, anti-CCL2 monoclonal antibodies are subcloned at least twice by limiting dilution.

Alternatively, antibodies may be isolated directly from DNA encoding the V_{H} and V_{L} domains of single antigen positive B cells from immunized transgenic mice (as described above) employing flow cytometry. Briefly, the human CCL2 immunized transgenic mice is terminated and splenocytes are harvested. Red blood cells are removed by lysis followed by pelleting the harvested splenocytes Resuspended splenocytes are first incubated with a cocktail of human IgG, FITC-anti-mFc, and biotinylated human CCL2 for 1 hour. The stained cells are washed twice with PBS, then stained with a cocktail of human and rat IgG, APC-anti-mIgM, and SA-PE for one hour. The stained cells are washed once with PBS and then analyzed by flow cytometry on a MOFLO™ XDP (Beckman Coulter, Inc.). Each IgG positive, IgM negative, and antigen positive B cell is sorted and plated into a separate well on a 96-well plate. RT-PCR of antibody genes from these B cells is performed according to a method described by Wang et al. (2000, J. Immunol. Methods 24-4:217-225). The heavy chain and light chain PCR products are cloned into vectors containing a human heavy chain constant region (e.g., IgG₁) and a human light chain constant region (e.g., Cκ), respectively. Purified recombinant plasmids having a heavy chain variable region sequence and plasmids having a light chain variable region sequence from the same B cell are then combined and transfected into a host cell line (e.g., a CHO cell line).

In addition to classic mouse immunization, other antibody screening methods based on camelids or phage display can also be used. High affinity antibodies are selected using standard receptor binding assays. Antibodies with affinity greater than 10⁻¹² M are purified.

### Example 2

### Dose Range Testing

This example illustrates a dose response study designed to evaluate effective dose ranges of anti-CCL2 antibody for treatment of scleroderma.

A bleomycin induced scleroderma mouse model is used in this example. Typically, fibrosis is induced in mice by repeated subcutaneous injection of bleomycin, polyinosinic-polycytidylic acid and/or LPS into the dorsal skin. Specifically, osmotic pumps (7-day) containing either bleomycin at concentration of 10-110 μg and up to 200 μg, LPS at a concentration of 300 μg, polycytidylic acid at a concentration of 100 μg or PBS alone are implanted subcutaneously into groups of 10 B6 mice. In this mouse model, histopathological changes in the skin closely resembles that seen in scleroderma. Early mononuclear cell accumulation and upregulated TGF-β and chemokine expression is followed by dermal fibrosis characterized by thick collagen bundles and accumulation of activated fibroblasts. Mice also manifest evidence of pulmonary and renal fibrosis.

Dose(s) of an anti-CCL2 antibody or a control antibody of escalating concentrations are administered into the mice via intraperitoneal injection.

### Example 3

### In Vive Efficacy of Anti-CCL2 Antibody

This example illustrates a study designed to evaluate the effect of treatment with anti-CCL2 antibodies on inflammation and fibrosis in the bleomycin mouse model for scleroderma.

7 or 28-day osmotic pumps containing either PBS alone or 10-110 pg and up to 200 pg bleomycin in PBS will be implanted subcutaneously into B6 mice. Every two days, mice will be treated via intraperitoneal injection with anti-CCL2 antibody at suitable concentrations, as determined in example 2, or with a control antibody.

After 7 days, in the case of a 7 day osmotic pump, or 28 days, in the case of a 28 day osmotic pump, skin and lung tissue will be harvested for transcriptional and histological analysis. Levels of CCL2 protein in tissue samples is measured by ELISA For transcriptional analysis, RNA is extracted from skin tissue and the isolated RNA is subject to and semi-quantitative or quantitative reverse transcriptase-PCR using techniques commonly known in the art. Levels of TGFβ gene expression and gene expression levels of pro-inflammatory genes, including but not limited to PAI1, COMP, COL1a1, F4/80, IL-6, and TNFα is measured using commercially available primers (TaqMan®) (TaqMan). For histological analysis, skin fibrosis is analyzed by microscopic examination of tissue sections stained with hematoxylin and eosin (H&E). The use of H&E staining to visualize tissue morphology is well known in the art. Immunohistochemistry is used to quantify monocyte infiltration by microscopic examination of tissue sections probed with the monocyte specific anti F4/80 antibody using techniques well known in the art.

It is anticipated that treatment with anti-CCL2 antibody will reduce infiltration of monocytes and macrophages, will reduce inflammatory gene expression (ex., IL-6, TNFα), and will decrease TGFβ-induced marker gene expression. This is expected to result in a general decrease in fibrosis.

### Example 4

### Therapeutic Modeling

This example illustrates a model of CCL2 production and turnover in various tissues and plasma to predict tissue target levels.

Typically, CCL2 is produced in disease tissues and secreted into plasma. In healthy individuals, CCL2 synthesis in skin is low or undetectable. CCL2 synthesis increases with involvement of total skin in both non-affected and affected skin, leading to increased serum CCL2 levels. Serum CCL2 levels further increase with organ involvement. Typically, healthy individuals have an average serum CCL2 level of less than about 100 pg/ml. Individuals having so called Raynaud's phenomenon have slightly increased average serum CCL2 levels. Patients suffering from sclerosis typically have an average serum CCL2 level of about 250 pg/ml. Patients suffering from limited cutaneous systemic sclerosis typically have an average serum CCL2 level of about 250 pg/ml. Patients suffering from diffuse cutaneous systemic sclerosis typically have an average serum CCL2 level of about 380 pg/ml. Patients suffering from limited cutaneous systemic sclerosis typically have an average serum CCL2 level of about 250 pg/ml.

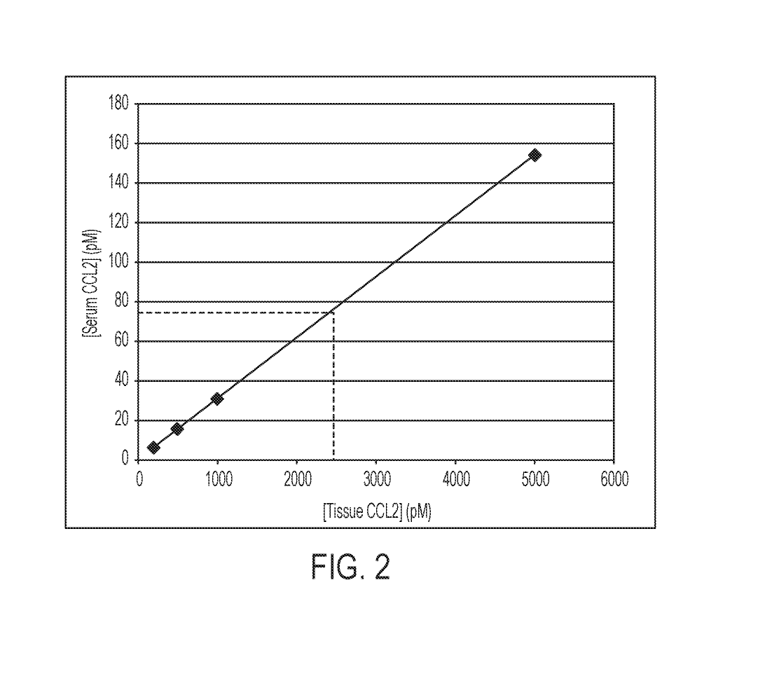

The molecular weight of CCL2 is about 8.6 kDa, which is much smaller than the glomerular filtration threshold of about 50 kDa, resulting in rapid kidney clearance. CCL2 is internalized by active receptor mediated internalization. Typical kd for CCL2 to bind its receptor CCR2 is about 60 pM-2 nM. CCR2 is primarily present on lymphoid-origin cells and lymphatic endothelium. It is contemplated that scleroderma causes increased vascular permeability early in disease progression, which permits substantial equilibration of CCL2 and any therapeutic antibodies between interstitium and serum. Therefore, serum half-life of CCL2 is about 10 minutes based on data from mice and rabbits. It is expected that CCL2 serum half-life in humans is similar. Relatively permeable tissue allows CCL2 reach equilibration from tissue to serum (half-max) quickly, for example in about 2 hours. In some cases, serum CCL2 level may reach 1000 pg/ml (˜75 pM) with whole skin involvement but without organ involvement. A target profile showing serum and tissue CCL2 equilibration is shown in FIG. 2, which predicts the desired amount of antibodies need to neutralize 3 nM of tissue CCL2 and competes it off its receptor. The illustrated model represents an extreme presentation of high CCL2 levels.

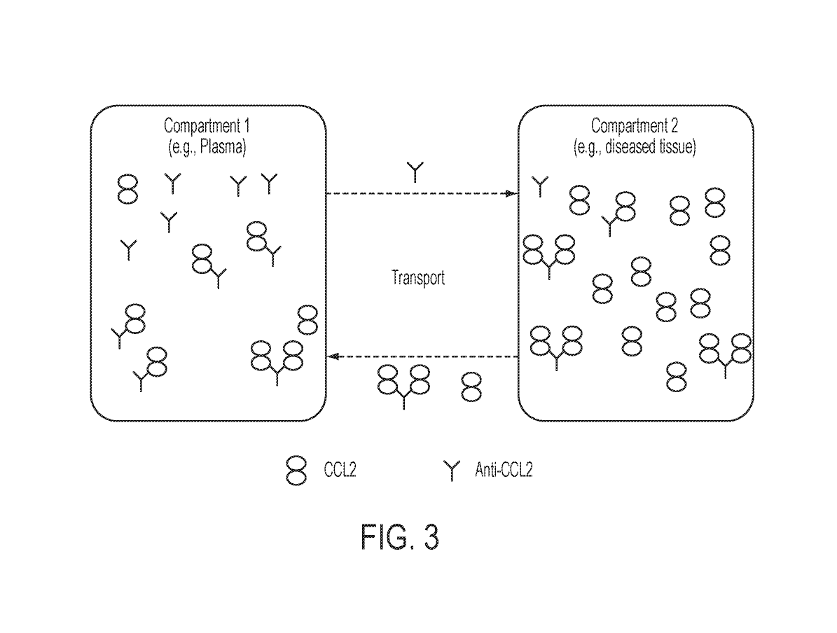

Currently available monoclonal antibodies injected intravenously typically are not effective because they bind CCL2 in plasma and forms a complex before they reach diseased tissues. See FIG. 3. By providing anti-CCL2 that is high-affinity, we can provide sufficient anti-CCL2 antibody to bind CCL2 in tissue and compete with the 60 pM affinity for CCR2.

### Example 5

### Clinical Design

Based upon the success of animal treatments, Phase I-III dose ranging and single dose studies of anti-CCL2 antibody detailed in Tables 2-6 are designed in healthy individuals and individuals with different stages of scleroderma to evaluate the safety, tolerability, efficacy, and pharmacokinetics of anti-CCL2 therapy.

A primary objective of Human Clinical Trial 1 includes determining the safety of 4 dose levels of anti-CCL2 antibody administered in healthy individuals. Secondary objectives include evaluating the pharmacokinetics of 4 different dose levels of anti-CCL2 antibody administered in healthy individuals. A detailed protocol synopsis of this clinical trial is shown in Table 2.

TABLE 2
Human Clinical Trial 1

| Phase | Phase 1 |
|---|---|
| # of Trials | 1 |
| Patient Population | Healthy volunteers |
| Trial Design and Endpoints | Single dose, dose escalation |
| | Primary: Safety |
| | Secondary: PK |
| # of Subjects | 4 dose groups |
| | n = 4 each |
| | 16 subjects total |
| Trial Length (FPI to LPV) | 0.5 years |
| | ~6 weeks to dose |
| | ~15 weeks follow up for PK |
| Comments | Single Phase 1 unit |

A primary objective of Human Clinical Trial 2 includes determining the safety of 4 dose levels of anti-CCL2 antibody administered in individuals with early symptoms of scleroderma. Secondary objectives include (1) to determine the pharmacokinetics of 4 different dose levels of anti-CCL2 antibody administered in individuals with early symptoms of scleroderma (2) to determine the pharmacodynamic (PD) response of individuals with early symptoms of scleroderma to 4 different dose levels of anti-CCL2 antibody by assaying gene expression in sequential skin biopsies and (3) to determine the clinical response of individuals with early symptoms of scleroderma to 4 different dose levels of anti-CCL2 antibody as measured by the Modified Rodnan Skin Score (mRSS). A detailed protocol synopsis of this clinical trial is shown in Table 3.

TABLE 3
Human Clinical Trial 2

| Phase | Phase 1/2 |
|---|---|
| # of Trials | 1 |
| Patient Population | Early (<2 yrs since non- |
| | Raynaud's Phenomenon |
| | (RP) symptom onset) diffuse SSc |
| | mRSS ≧ 15 |
| Trial Design and Endpoints | Multiple Dose Escalation |
| | Double-blind placebo-controlled |
| | Treatment duration: 6 months |
| | 4 Dose levels |
| | Primary: Safety |
| | Secondary: PK |
| | PD response (sequential skin biopsy gene |
| | expression - baseline, 4 wks, 6 months) |
| | Clinical response (mRSS) |
| # of Subjects | 4 dose groups |
| | n = 10 each (8 active/2 placebo) |
| | 40 subjects total |
| Trial Length (FPI to LPV) | 1.5 years |
| Comments | Up to 8 sites to recruit within 1 yr |

A primary objective of Human Clinical Trial 3 includes determining the efficacy of a single dose level of anti-CCL2 antibody administered in individuals with early symptoms of scleroderma as measured by the Modified Rodnan Skin Score (mRSS). Secondary objectives include (I) determining the efficacy of a single dose level of anti-CCL2 antibody administered in individuals with early symptoms of scleroderma as measured by the Health Assessment Questionnaire-Disability Index (HAQ-DI) and (2) determining the efficacy of a single dose level of anti-CCL2 antibody administered in individuals with early symptoms of scleroderma as measured by organ specific assessments. A detailed protocol synopsis of this clinical trial is shown in Table 4.

TABLE 4
Human Clinical Trial 3

| Phase | Phase 2 |
|---|---|
| # of Trials | 1 |
| Patient Population | Early (<2 yrs since non- |
| | Raynaud's Phenomenon |
| | (RP) symptom onset) diffuse SSc |
| | mRSS ≧ 15 |
| Trial Design and Endpoints | 1 dose level |
| | Double-blind Placebo Controlled |
| | Parallel Group |
| | Treatment duration 6 months |
| | Open-label extension |
| | Primary: mRSS |
| | Secondary: HAQ DI, organ-specific |
| | assessments |
| # of Subjects | 2:1 randomization |
| | 120 subjects total |
| Trial Length (FPI to LPV) | 1.5 years |
| Comments | Up to 20 sites to recruit within 1 yr |

A primary objective of Human Clinical Trial 4 includes determining the efficacy relative to oral cyclophosphamide of a single dose level of anti-CCL2 antibody administered in individuals with limited or diffuse scleroderma with lung disease as measured by Forced Vital Capacity (FVC). Secondary objectives include (1) determining the efficacy relative to oral cyclophosphamide of a single dose level of anti-CCL2 antibody administered in individuals with limited or diffuse scleroderma with lung disease as measured by the HAQ-DI, (2) determining the efficacy relative to oral cyclophosphamide of a single dose level of anti-CCL2 antibody administered in individuals with limited or diffuse scleroderma with lung disease as measured by the mRSS, and (3) determining the efficacy relative to oral cyclophosphamide of a single dose level of anti-CCL2 antibody administered in individuals with limited or diffuse scleroderma with lung disease as measured by diffusing capacity of the lung for carbon monoxide (DLCO). A detailed protocol synopsis of this clinical trial is shown in Table 5.

TABLE 5
Human Clinical Trial 4

| Phase | Phase 2 |
|---|---|
| # of Trials | 1 |
| Patient Population | Limited or Diffuse SSc with lung disease: |
| | Active alveolitis by HRCT |
| | <7 yrs since non-RP symptom onset |
| | FVC < 85% > 45% predicted |
| Trial Design and Endpoints | 1 dose level |
| | Double-blind Controlled Parallel Group |
| | Comparator: SoC (oral cyclophosphamide) |
| | Treatment duration 12 months |
| | Open-label extension |
| | Primary: FVC |
| | Secondary: DLCO, HAQ DI, mRSS |
| # of Subjects | 2:1 randomization |
| | 120 subjects total |
| Trial Length (FPI to LPV) | 1.5 years |
| Comments | Up to 10 sites to recruit within 6 months |

Objective of Human Clinical Trial 5 include (1) determining the efficacy relative to oral cyclophosphamide of a single dose level of anti-CCL2 antibody administered in individuals with early symptoms of scleroderma and/or limited or diffuse scleroderma with lung disease as measured by Forced Vital Capacity (FVC), (2) determining the efficacy relative to oral cyclophosphamide of a single dose level of anti-CCL2 antibody administered in individuals with early symptoms of scleroderma and/or limited or diffuse scleroderma with lung disease as measured by the HAQ-DI, (3) determining the efficacy relative to oral cyclophosphamide of a single dose level of anti-CCL2 antibody administered in individuals with early symptoms of scleroderma and/or limited or diffuse scleroderma with lung disease as measured by mRSS, and (4) determining the efficacy relative to oral cyclophosphamide of a single dose level of anti-CCL2 antibody administered in individuals with early symptoms of scleroderma and/or limited or diffuse scleroderma with lung disease as measured by DLCO. A detailed protocol synopsis of this clinical trial is shown in Table 6.

TABLE 6
Human Clinical Trial 5

| Phase | Phase 3 |
|---|---|
| # of Trials | 1 each |
| Trial Design and Endpoints | Single dose level, double-blind head-to- head |
| | comparison with SoC in either or both early |
| | dSSc or SSc Lung Disease, depending on |
| | outcome of Phase 2s |
| | Endpoints as in Phase 2 |
| # of Subjects | 120 patients each |
| Trial Length (FPI to LPV) | 2.0 years |
| | 0.5 to 1 year enrollment period |
| Comments | Treatment duration 12 months |

Patients exhibiting early symptoms of scleroderma treated with anti-CCL2 antibody are expected to demonstrate significant improvement of symptoms as measured by the mRSS and HAQ-DI. Patients with limited or diffuse scleroderma with lung disease treated with anti-CCL2 antibody are expected to demonstrate significant improvement of symptoms as measured by the mRSS, HAQ-DI, and FVC. Anti-CCL2 antibody is expected to be more effective than cyclophosphamide in treatment of patients either with early symptoms of scleroderma or with limited or diffuse scleroderma with lung disease as measured by mRSS, HAQ-DI, and/or FVC.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a”, “an” and “the” may mean one or more than one unless indicated to the contrary or otherwise evident from the context. Thus, for example, reference to “an antibody” includes a plurality of such antibodies, and reference to “the cell” includes reference to one or more cells known to those skilled in the an, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are presenting, employed in, or otherwise relevant to a given product or process. Furthermore, it is to be understood that the invention encompasses all variations, combinations, and permutations in which one or more limitation, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for anyone of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understand of one of ordinary skill in the art, values that are expressed as ranges can assume any specific value or sub-range within the state ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any HCV genotype/subtype, any HCV antibody, any epitope, any pharmaceutical composition, any method of administration, any therapeutic application, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

### OTHER EMBODIMENTS

Those of ordinary skill in the art will readily appreciate that the foregoing represents merely certain preferred embodiments of the invention. Various changes and modifications to the procedures and compositions described above can be made without departing from the spirit or scope of the present invention, as set forth in the following claims.

1. A method of treating scleroderma comprising
administering to an individual who is suffering from or susceptible to scleroderma an effective amount of anti-CCL2 antibody, or fragment thereof, such that at least one symptom or feature of scleroderma in a target tissue is reduced in intensity, severity, or frequency, or has delayed onset.

2. The method of claim 1, wherein the at least one symptom or feature of scleroderma is selected from endothelial-cell damage, proliferation of basal-lamina layers, perivascular mononuclear-cell infiltration, fibrosis, derangement of visceral-organ architecture, rarefaction of blood vessels, hypoxia, and combination thereof.

3. The method of claim 1, wherein the target tissue is selected from the group consisting of skin, blood vessels, lung, heart, kidney, gastrointestinal tract (including liver), musculoskeletal system and combinations thereof.

4. (canceled)

5. (canceled)

6. The method of claim 1, wherein the individual is suffering from or susceptible to limited cutaneous scleroderma.

7. The method of claim 1, wherein the individual is suffering from or susceptible to diffuse cutaneous scleroderma.

8. The method of claim 1, wherein the anti-CCL2 antibody, or fragment thereof, is administered parenterally.

9. The method of claim 8, wherein the parenteral administration is selected from intravenous, intradermal, inhalation, transdermal (topical), subcutaneous, and/or transmucosal administration.

10. The method of claim 9, wherein the parenteral administration is intravenous administration.

11. The method of claim 1, wherein the anti-CCL2 antibody, or fragment thereof, is administered orally.

12. The method of claim 1, wherein the anti-CCL2 antibody, or fragment thereof, is administered bimonthly, monthly, triweekly, biweekly, weekly, daily, or at variable intervals.

13. A method of treating scleroderma comprising
administering to an individual who is suffering from or susceptible to scleroderma an anti-CCL2 antibody, or fragment thereof, having a binding affinity of greater than 10⁻¹² M.

14. The method of claim 13, wherein the anti-CCL2 antibody, or fragment thereof, is administered at a therapeutically effective dose and an administration interval such that the anti-CCL2 antibody, or fragment thereof, is distributed to one or more target tissues selected from the group consisting of skin, blood vessels, lung, heart, kidney, gastrointestinal tract (including liver), musculoskeletal system and combinations thereof.

15. The method of claim 13, wherein the anti-CCL2 antibody, or fragment thereof, is administered at a therapeutically effective dose and an administration interval such that the anti-CCL2 antibody, or fragment thereof, is distributed to lung and/or heart.

16. (canceled)

17. A method of treating scleroderma comprising
administering to an individual who is suffering from or susceptible to scleroderma an anti-CCL2 antibody, or fragment thereof, at a therapeutically effective dose and an administration interval such that the anti-CCL2 antibody, or fragment thereof, is distributed to lung and/or heart.

18. The method of claim 17, wherein the anti-CCL2 antibody, or fragment thereof, is further distributed to skin, kidney, and/or liver.

19. The method of claim 17, wherein the anti-CCL2 antibody, or fragment thereof, is selected from the group consisting of intact IgG, F(ab′)₂, F(ab)₂, Fab′, Fab, scFvs, diabodies, triabodies and tetrabodies.

20. The method of claim 19, wherein the anti-CCL2 antibody, or fragment thereof, is a monoclonal antibody.

21. The method of claim 20, wherein the anti-CCL2 antibody, or fragment thereof, is a humanized monoclonal antibody.

22. The method of claim 20, wherein the anti-CCL2 antibody, or fragment thereof, is a human antibody.

23. An anti-CCL2 antibody, or fragment thereof, having a binding affinity of greater than 10⁻¹² M.

24. The anti-CCL2 antibody of claim 23, wherein the anti-CCL2 antibody, or fragment thereof, has a binding affinity of greater than 10⁻¹³ M.

25. The anti-CCL2 antibody of claim 23, wherein the anti-CCL2 antibody, or fragment thereof, is selected from the group consisting of intact IgG, F(ab′)2, F(ab)2, Fab′, Fab, scFvs, diabodies, triabodies and tetrabodies.

26. The anti-CCL2 antibody claim 23, wherein the anti-CCL2 antibody, or fragment thereof, is a monoclonal antibody.

27. (canceled)

28. (canceled)

29. A kit comprising an anti-CCL2 antibody, or fragment thereof, according to claim 23.


SEQUENCE LISTING (1 sequence)

SEQ ID NO: 1 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT